SEQUENCE LISTING (5 sequences)

SEQ ID NO: 1 - protein, 28 aa, Solanum tuberosum
MASSATTKSFLILFFMILATTSSTCAVD

SEQ ID NO: 2 - protein, 18 aa, Homo sapiens
MKWVTFISLLFLFSSAYS

SEQ ID NO: 3 - protein, 19 aa, Homo sapiens
MTRLTVLALLAGLLASSRA

SEQ ID NO: 4 - protein, 25 aa, Cricetulus griseus
MASSGSVQQPRLVLLMLVLAGAARA

SEQ ID NO: 5 - protein, 23 aa, Cricetulus griseus
MAPFASLASGILLLLSLITSSKA

(19) United States
(12) Patent Application Publication
(10) Pub. No.: US 2024/0277802 A1
(43) Pub. Date: Aug. 22, 2024

(54) Materials and Methods for Therapy of the Musculoskeletal System and Other Tissues

(71) Applicant: BioStrategies LC, State University, AR (US)

(72) Inventors: Walter ACOSTA, State University, AR (US); Carole L. CRAMER, Jonesboro, AR (US); Tianhong YANG, State University, AR (US)

(21) Appl. No.: 18/583,271

(22) Filed: Feb. 21, 2024

Related U.S. Application Data
(60) Provisional application No. 63/486,143, filed on Feb. 21, 2023.

(51) Int. Cl. A61K 38/17; A61K 48/00; A61P 3/00; C12N 15/86

(52) U.S. Cl. CPC A61K 38/1732; A61K 48/005; A61P 3/00; C12N 15/86; C12N 2750/14143

(57) ABSTRACT

The subject invention provides materials, compositions, and methods for treating diseases or injury of, or involving skeletal, muscular or connective tissues in a subject. Compounds/fusions and compositions of the invention utilize plant lectins for delivery of therapeutic agents and/or other associated molecules to cells, tissue, and/or organs of the musculoskeletal system including bone, muscle, cartilage, and/or connective tissue.

### CROSS-REFERENCE TO RELATED APPLICATION

This application claims priority to U.S. Provisional Patent Application No. 63/486,143, filed Feb. 21, 2023, which is incorporated herein by reference in its entirety.

### SEQUENCE LISTING

The Sequence Listing for this application is labeled “SeqList—as filed.xml,” which was created on Feb. 20, 2024, and is 5,273 bytes. The Sequence Listing is incorporated herein by reference in its entirety.

### BACKGROUND OF THE INVENTION

As the collective experience of medical professionals and patients receiving long-term treatments with enzyme replacement therapies or other protein-based therapeutics expands, so called “hard to treat” cells, tissues and organs have emerged as being particularly difficult to which to deliver corrective doses. These include the brain and central nervous system and the bone and other components of the musculoskeletal system including muscle, joints, cartilage, tendons and other structural support tissues.

Bone is particularly challenging to treat due to its developmental pattern. Rapid growth occurs at various developmental stages and bone strength and quality is maintained by a continuous breakdown and regeneration cycle (bone remodeling). Many lysosomal diseases have significant musculoskeletal system involvement leading to, for example, dwarfism, brittle bone, dysostosis multiplex, cervical stenosis, kyphoscoliosis and facial/physical deformities.

Mucopolysaccharide (MPS) type I (Hurler; Hurler/Scheie; Scheie syndromes) and type IVA (Morquio disease) are two dramatic examples. MPS IVA is a chronic, progressive lysosomal storage disorder. It is caused by a deficiency of N-acetyl-galactosamine-6-sulfate sulfatase (GALNS) resulting in pathogenic accumulation of the glycosaminoglycans (GAG), keratan sulfate (KS) and chondroitin 6-sulfate (C6S) in many tissues and organs. Failure to effectively clear GAG leads to clinical manifestations affecting the heart, visceral organs, eyes, respiratory system, brain, bones, and joints. Skeletal disease limits patient's mobility due to the development of systemic skeletal dysplasia, short stature, and joint abnormalities. Patients require physiotherapy, multiple surgical interventions (most commonly spine, hips, trachea), and are often wheelchair dependent in their second decade of life^{1,22,3}.

Both MPS I and IVA have approved enzyme replacement therapies (ERTs) available for disease treatment. However, with long-term therapy, significant and debilitating skeletal abnormalities develop, indicating that enzyme treatment is not effectively delivered to the critical skeletal sites that have accumulated pathological levels of GAGs, the disease substrates that is elevated in MPS patients.

An ERT for MPS IVA, elosulfase alfa (Vimizin®), was FDA-approved for use in the US in 2014, but provides only limited effectiveness in supporting bone growth and reducing dysplasia^{2,4-6}. Beyond the huge burdens on patient families, costs to the health care system run as much as $800,000 annually.

Vimizim is a recombinant human GALNS produced in CHO cells. Cell uptake and lysosomal delivery of Vimizim are based on the interaction of GALNS glycoprotein with mannose-6-phosphate receptors (M6PR) on target cells. M6PR-mediated uptake is the dominant approach used for lysosomal ERTs and supports the improvement of visceral organs; however, drug access to some tissues (e.g., bone; brain) is inadequate. M6PR, which primarily functions intracellularly to shuttle lysosomal enzymes from the ER to lysosomes, displays low abundance on the plasma membrane of most cell types and is limited or absent in so-called hard-to-treat tissues such as the brain endothelium⁷.

A second limitation of the current replacement enzymes is the development of anti-drug antibodies (ADA) in patients. Mechanistically, ADAs block M6PR-mediated drug uptake and can redirect the drug to cells of the immune system⁸⁻¹⁰, changing its biodistribution, thereby limiting pathology correction in key tissues. In Phase III clinical trials, 100% of Morquio A patients treated with Vimizin developed antibodies against the enzyme^{2,11,12}. In addition, all patients tested positive for neutralizing antibodies, capable of interfering with M6PR during the study, and most remained ADA positive afterward (67-92%)^{13,14}.

Further, there are additional genetic disorders leading to severe bone, muscle and cartilage pathologies. This includes both the dominant and recessive forms of osteogenesis imperfecta and glycogen storage disorders, such as Pompe, for which no effective treatment exists.

Thus, there is significant need to develop carrier systems that are capable of effectively delivering enzymes and other therapeutic agents at corrective doses to these “hard-to-treat” sites within the musculoskeletal system.

### BRIEF SUMMARY OF THE INVENTION

The subject invention provides materials and methods for treating diseases, or injury of, or involving, skeletal, muscular, or connective tissues in a subject. Compounds and compositions of the invention utilize lectins for delivery of therapeutic agents and/or other associated molecules to cells, tissue, and/or organs of the musculoskeletal system including bone, muscle, cartilage, and/or connective tissue (e.g., cells of mesenchymal origin).

In one embodiment, the subject invention provides a fusion protein comprising a lectin and a therapeutic agent, wherein the lectin is fused to the therapeutic agent via a linker or a spacer sequence of amino acids. In certain embodiments, the fusion comprises a therapeutic agent connected directly to a lectin.

In specific embodiments, the subject invention provides a fusion protein comprising a plant lectin and a therapeutic protein or peptide, wherein the plant lectin is fused to the therapeutic protein or peptide via a linker or a spacer sequence of amino acids. In certain embodiments, the fusion protein comprises a therapeutic protein connected directly to a lectin.

In certain embodiments, the therapeutic proteins are selected from enzymes, proteases, antibodies, chaperones, and growth factors. In specific embodiments, the therapeutic protein is selected from CAPN3, GYS1, POGLUT1, GAA, PYGM, AGL, GBE1, PYGL, PFKM, ALDOA, GYS2, ENO3, DSE, CHST14, PGAM2, GTG1, FKRP, POMT1, CAPN3, NEU1, SMPD1, IDS, NAGLU, SGSH, IDUA, GNS, GALNS, ARSB, HYAL1, MAN2B1, FUCA1, NAGA, P3H1, FKBP10, BMP1, WNT1, SPARC, MESD, CRTAP, SERPINH1, SERPINF1 and TENT5A.

In certain embodiments, the plant lectin is selected from 1) B subunits from AB toxins such as ricins, abrins, nigrins, and mistletoe toxins, viscumin toxins, ebulins, pharatoxin, hurin, phasin, and pulchellin; and 2) wheat germ agglutinin, peanut agglutinin, and tomato lectin. In a preferred embodiment, the plant lectin is RTB or NNB.

In specific embodiments, the fusion protein comprises a therapeutic protein fused to RTB. Preferably, the therapeutic protein is selected from IDUA, GALNS, GAA, CRTAP, P3H1, beta-Gal, and ARSB.

In specific embodiments, the fusion protein is IDUA fused to RTB, GALNS fused to RTB, GAA fused to RTB, CRTAP fused to RTB, P3H1 fused to RTB, BetaGal fused to RTB and ARSB fused to RTB.

In one embodiment, the fusion protein is produced in plant, fungal, bacterial, mammalian or human cells or organisms (e.g., patient organisms).

In certain embodiments, the subject invention provides a fusion entity comprising a first component responsible for delivering a second component to cells, wherein the first component is selected from plant lectins, and fragments thereof, and the second component is a therapeutic agent selected from, for example, small molecules, proteins, peptides, and nucleic acids. In one embodiment, the first component is operatively associated with the second component.

In certain embodiments, the therapeutic agent comprises genetic materials such as nucleic acids (e.g., RNA, DNA, cDNA, etc.) that provide for gene therapy and/or virus-assisted gene transfer, such that a therapeutic protein or peptide can be synthesized de novo within the cells of a treated subject or can be synthesized within cells that can then be used as a cell-based therapy for a subject.

In one embodiment, the subject invention provides polynucleotide sequences encoding the fusion proteins of the subject invention.

In one embodiment, the subject invention provides a composition comprising a fusion protein of the subject invention or the polynucleotide sequence encoding a fusion protein of the subject invention.

In one embodiment, the subject invention provides a cell comprising a polynucleotide sequence encoding a fusion protein of the subject invention or a cell expressing a fusion protein of the subject invention.

In one embodiment, the subject invention provides a method for treating a musculoskeletal condition, the method comprising administering, to a subject in need thereof, (i) a fusion construct comprising a lectin and a therapeutic agent, (ii) a polynucleotide sequence encoding the fusion construct comprising the lectin and the therapeutic agent, (iii) a cell comprising the polynucleotide sequence encoding the fusion construct comprising the lectin and the therapeutic agent, or (iv) a cell expressing the fusion construction comprising the lectin and the therapeutic agent.

In specific embodiments, the musculoskeletal condition is selected from genetic diseases with skeletal pathology, lysosomal diseases with skeletal pathology, collagen-associated diseases, bone diseases, and genetic muscle diseases.

In some embodiments, the fusion construct is produced in plant, fungal, bacterial cells, mammalian cells or organisms.

In one embodiment, the method for treating a musculoskeletal condition comprises administering, to a subject in need thereof, a) a fusion construct comprising a lectin and a therapeutic agent, or b) a polynucleotide sequence encoding a fusion construct comprising a lectin and a therapeutic agent.

In one embodiment, the subject invention provides a method for treating a musculoskeletal condition, the method comprising administering, to a subject in need thereof, a composition comprising a) a fusion construct comprising a lectin and a therapeutic agent, or b) a polynucleotide sequence encoding a fusion construct comprising a lectin and a therapeutic agent.

In certain embodiments, the subject invention provides methods for treating a musculoskeletal condition in a subject, the method comprising administering the fusion protein, the polynucleotide sequence encoding the fusion protein, or the composition, of the subject invention, to the subject in need thereof, wherein the administration, for example, is oral, subcutaneous, intrajoint, intradermal, intravenous, intramuscular, intraperitoneal, or intrasternal administration.

In some embodiments, the polynucleotide sequence encoding the fusion protein comprising the plant lectin and the therapeutic protein can be used as a gene therapy, such as, an adeno-associated virus (AAV) vector gene therapy.

In certain embodiments, the subject invention provides methods for treating a musculoskeletal condition in a subject, the method comprising administering, to the subject in need thereof, a cell comprising the polynucleotide sequence encoding a fusion protein of the subject invention or a cell expressing the fusion protein of the subject invention.

In certain embodiments, the subject invention provides a method for treating a musculoskeletal condition in a subject, the method comprising administering, to the subject in need thereof, a gene therapy vector, such as, an AAV gene therapy vector, wherein the AAV gene therapy vector comprises a nucleic acid sequence encoding a lectin and a nucleic acid sequence encoding a therapeutic protein.

In some embodiments, the musculoskeletal condition is selected from genetic diseases with skeletal pathology, lysosomal diseases with skeletal pathology, collagen-associated diseases, bone diseases, and genetic muscle diseases. In specific embodiments, the musculoskeletal disease is selected from Muscular Dystrophy, Limb-Girdle, Autosomal Recessive 1; Glycogen Storage Disease (e.g., Pompe Diseases); Muscular Dystrophy, Limb-Girdle, Autosomal Recessive 21; Ehlers-Danlos Syndrome; Muscular Dystrophy-Dystroglycanopathy; Autosomal Recessive Limb-Girdle Muscular Dystrophy Type 2a; Neuraminidase Deficiency; Niemann-Pick Disease, Type B; Mucopolysaccharidosis type I; Mucopolysaccharidosis type II; Mucopolysaccharidosis type IIIA; Mucopolysaccharidosis type IIIB; Mucopolysaccharidosis type IIID; Mucopolysaccharidosis type IVA; Mucopolysaccharidosis type VI; Mucopolysaccharidosis type IX; Mannosidosis, Alpha B, Lysosomal; Fucosidosis; Schindler Disease, Type I; and Osteogenesis Imperfecta.

In a preferred embodiment, the lysosomal diseases are selected from MPS I, MPS IVA, MPS IVB, MPS VI, and Pompe Diseases.

In one embodiment, the subject invention provides a method for delivering a therapeutic agent to cells of the musculoskeletal system of a subject, the method comprising administering, to the subject, (i) a fusion construct comprising a lectin and a therapeutic agent, or (ii) a polynucleotide sequence encoding the fusion construct comprising the lectin and the therapeutic agent.

In one embodiment, the subject invention further provides a method for delivering a therapeutic agent to cells of the musculoskeletal system of a subject, the method comprising administering a fusion protein comprising a plant lectin and a therapeutic agent to the subject in need, or administering a composition comprising the fusion protein that comprises a plant lectin and a therapeutic agent to the subject in need.

In a specific embodiment, the fusion protein is selected from IDUA:RTB, RTB:GALNS, GAA:RTB, CRTAP:RTB, P3H1:RTB, beta-Gal:RTB, and ARSB:RTB.

One aspect of the subject invention provides a plant galactose/galactosamine-binding lectin, for example, ricin B chain (RTB), as a unique enzyme carrier for genetically fused human lysosomal enzymes. Advantageously, RTB has a remarkable ability to deliver fused enzymes to lysosomes of the classic “hard-to-treat” cells, tissues and organs including bone, connective tissues, chondrocytes, skeletal muscle, and heart.

In MPS I mice, long-term treatments with RTB-delivered ERTs showed bone normalization during both growth and remodeling phases based on micro-CT scanning. Further, RTB delivers fused enzymes into cells and hard-to-treat tissues even in the presence of neutralizing anti-enzyme antisera (ADA). Thus, an RTB carrier module of the invention can be used to address the most significant limitations and unmet medical needs for other lysosomal and metabolic disorders—efficient delivery to bone and connective tissues, the ability to improve skeletal readouts during bone growth and bone remodeling phases, and the potential to mitigate ADA immune responses.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIG. 1. RTB trafficking.

FIG. 2. IDUA:RTB uptake saturation in MPS I fibroblasts. Intracellular IDUA activity after 4 h incubation with IDUA:RTB or med-IDUA (mammalian cell-derived IDUA; R&D Systems) at concentrations of 0.9-30 μM.

FIGS. 3A-3C. Efficacy of long-term administration of IDUA:RTB. MPS I mice (7-8 weeks old; n=12) were treated weckly with IDUA:RTB at 0.58 or 1.0 mg IDUA equivalents/kg BW and compared with mock-treated MPS I or WT (Het siblings). 3A) GAG levels were quantified in tissues 13 d after 8th injection. 3B) LAMP-2 immunostaining of the hippocampal region. MPS I mouse brains were processed by sagittal sections cryo-sliced in 20 μM. Immunohistochemistry was performed using rat α-LAMP2 (lysosomal compartment), goat α-rat Cy5 and counterstained with DAPI (nuclei). 3C) High-content imaging of cerebral sections comparing lysosome compartment area. 6-8 sagittal slices per animal were systematically mounted in the slide. Entire area of all slices was scanned at 4× using an automated microscope. LAMP-2 signal/area of tissue was calculated for each slice (n=6-8). Each bar represents combined data for a single animal. Tukey multiple comparison analysis demonstrate significant difference of treatment groups compared to untreated mice (p<0.0009).

FIGS. 4A-4C. RTB:EGFP uptake into human cells of the mesenchyme lineage. 4A) Chondrocytes of cartilage plate of human lung (t=120 m; monochrome image, R. Kurten, UAMS). 4B) Mesenchymal stem cells (t=75 m); time-lapse video capture so reduced resolution (1. Suva, UAMS). 4C) Fibroblasts (t=60 m).

FIG. 5: Expression levels of Mmp3 in MPS I mice treated with IDUA:RTB. **p<0.01***p<0.001

FIGS. 6A-6E: Correction of Bone Pathology in MPS I mice treated with IDUA:RTB starting at 3 weeks of age. 6A) Animal performance video-recorded on an open field test at 23 weeks of age (n=6). Total distance travelled (left), and the maximum speed (middle) achieved by the subject were calculated by a tracking software (Anymaze). Percentage of change of maximum speed between first and third trial (right). 6B) Micro-CT architecture in tibias (n=6). 6C) Mono-sulfated keratan sulfate concentration in serum (n=6). 6D) Volcano plot of gene expression changes in femur transcriptome after 24 weeks treatment with 0.58 mg/kg of IDUA RTB. 6E) Reduction of lysosomal deposit in chondrocytes of the growth plate. Tibia sections were stained with toluidine blue for histopathology analysis. GAG deposition is noted in chondrocytes of untreated IDUA^{-/-} (KO) mice at 24 weeks of age (white arrows). Aged matched Normal (IDUA−/+) or KO mice treated with 0.58 mg/kg of IDUA:RTB shows no deposit of GAG within chondrocytes.

FIG. 7: Correction of Bone Pathology in MPS I mice treated with IDUA:RTB starting at 8 weeks of age.

FIG. 8. IDUA:RTB distributes to lysosomes of skeletal muscle tissues. Infrared-fluorescence-labeled IDUA:RTB or med-IDUA (Aldurazyme equivalent) was administrated to MPS I mice by tail-vein injection (2 mg/kg). At 24 hr, isolated muscle tissue was stained with DAPI (nuclei) and imaged. Only IDUA:RTB is seen as red punctate intracellular structures consistent with lysosomal delivery.

FIGS. 9A-9C. Impact of ADA on delivery of IDUA:RTB. 9A) Enzyme activity in organs of immunized or naïve mice injected with 2 mg mcd-IDUA equivalents/kg of IDUA:RTB. 9B) Fluorescent intensity in bones from mice injected with fluorescent labelled IDUA:RTB (n=5). Untreated control mice were injected with equivalent mols of unreactive dye (IRDye800CW carboxylate). 9C) Correlation of therapeutic response and ADA in MPS I mice treated weekly or biweekly with 1 mg/Kg of IDUA:RTB.

FIG. 10. RTB:GALNS purity. Coomassie gel comparing fractions after lactose affinity & size exclusion chromatography (SEC). Western blot using aRTB antibody in the far-right lane.

FIGS. 11A-11B. RTB:GALNS uptake. 11A) Uptake capacity assessed in MPS IVA patient's fibroblast by measuring intracellular GALNS activity after 24 h incubation with protein at media concentrations of 25-150 nM. 11B) Intracellular processing. Lysates immunoblotted with anti-GALNS antibodies (MyBiosource #MBS1750805) after 24 h incubation with media containing RTB:GALNS.

FIG. 12. Correction of lysosomal phenotype in galns^{+/-} fibroblasts.

FIG. 13. Ex-vivo biodistribution. % of fluorescence in each organ 24 h after infusion of fluorescently labelled RTB:GALNS, IDUA:RTB, or Vimizin. Eight weeks old mice were treated with 2 mg/kg of RTB:GALNS⁶⁸⁰, IDUA:RTB⁶⁸⁰ or Vimizin⁶⁸⁰ and harvested 24 h after infusion

FIG. 14. Biodistribution to bone+/−BM (bone marrow).

FIG. 15. RTB:eGFP uptake in human Morquio A patient-chondrocytes.

FIG. 16. Expression of GAA:RTB in crude extracts from plant leaves 2 to 5 days after transfection.

FIGS. 17A-17B. Cell uptake and intracellular processing of GAA8 in human Pompe fibroblast cells. 17A) GAA8 showed greater cell uptake and higher saturation than recombinant human α-GAA (rhGAA) in Pompe fibroblasts. 17B) Pompe fibroblasts treated with 50 nM of GAA8 were harvested daily for up to 8 days. Cells lysates were analyzed by western blot detected with anti-GAA antibodies. The processed GAA8 (shown as 76 kDa in size) is stable up to 8 days in Pompe fibroblasts.

FIG. 18. GAA8 treated Pompe mice showed significant glycogen reductions in muscle. Five mg/kg of rhGAA or equivalent molar dose of GAA8 were weekly administrated in 8-wks-old Pompe mice via tail vein injections for 4 weeks. (n=3 mice in each treatment group). Error bars represent means±SD. *p<0.05, **p<0.01, ***p<0.001, ****p<0.0001 between groups.

FIGS. 19A-19C. RTB^{ER} directs C3/P1 complex to the ER of CRTAP^{-/-} fibroblasts (19A and 19B) and C3 to osteoblasts (19C) based on co-localization (yellow) with ER markers.

### BRIEF DESCRIPTION OF SEQUENCES

SEQ ID NO: 1 is a modified patatin signal sequence contemplated for use according to the subject invention.

SEQ ID NO: 2 is an albumin signal sequence contemplated for use according to the subject invention.

SEQ ID NO: 3 is an Azurocidin signal sequence contemplated for use according to the subject invention.

SEQ ID NO: 4 is a Cathepsin Z signal sequence contemplated for use according to the subject invention.

SEQ ID NO: 5 is a Metalloproteinase inhibitor 1 signal sequence contemplated for use according to the subject invention.

### DETAILED DESCRIPTION OF THE INVENTION

The subject invention provides materials and methods for treating diseases, or injury of, or involving, skeletal or connective tissues in a subject. Compounds and compositions of the invention utilize lectins (e.g., plant lectins) for delivery of therapeutic agents and/or other associated molecules to cells, tissue, and/or organs of the musculoskeletal system including bone, muscle, cartilage, and/or connective tissue (e.g., cells of mesenchymal origin).

Compounds and compositions of the invention can be designed to deliver a therapeutic agent intracellularly, for example, to lysosome and/or to endoplasmic reticulum (ER) following cell uptake in musculoskeletal tissue.

In one embodiment, the subject invention provides a compound, complex, conjugate or fusion entity comprising a first component responsible for delivering a second component to cells, wherein the first component is selected from lectins, and fragments thereof, and the second component is a therapeutic agent selected from, for example, small molecules, proteins, peptides, and nucleic acids. In one embodiment, the first component is operatively associated with the second component.

In specific embodiments, the therapeutic agent is a protein selected from, for example, antiproteases, enzymes, antibodies, structural proteins, coagulase factors, interleukins, cytokines, growth factors, interferons, and lymphokines.

In certain embodiments, the lectin is selected from lectins found in plants, microorganisms, and animals. In specific embodiments, the lectin is selected from, for example, bacterial lectins (e.g., Shiga toxin B, cholera toxin B, and pertussis B subunit), amoebal lectins, and viral lectins (e.g., influenza viral lectin). In specific embodiments, the lectin is selected from animal lectins that include, but are not limited to, calnexin, calreticulin, ERGIC-53, collectins, dectin-1, galectins, macrophage mannose receptor, man-6-P receptors, L-selectin, E- and P-selectins, Siglecs, and Spermadhesin.

In one embodiment, the subject invention provides a fusion protein comprising a first component selected from plant lectins and a second component that is a therapeutic protein or peptide. In certain embodiments, the therapeutic protein is selected from enzymes, proteases, chaperones, and growth factors. In specific embodiments, the therapeutic protein is selected from CAPN3, GYS1, POGLUT1, GAA, PYGM, AGL, GBE1, PYGL, PFKM, ALDOA, GYS2, ENO3, DSE, CHST14, PGAM2, GTG1, FKRP, POMT1, CAPN3, NEU1, SMPD1, IDS, NAGLU, SGSH, IDUA, GNS, GALNS, ARSB, HYAL1, MAN2B1, FUCA1, NAGA, P3H1, FKBP10, BMP1, WNT1, SPARC, MESD, CRTAP, SERPINH1, SERPINF1 and TENT5A.

Therapeutic agents within the scope of the invention include therapeutic proteins directed to diseases with musculoskeletal pathology including genetic or lysosomal storage diseases. Also contemplated are therapeutic proteins that are directed to therapy of collagen-associated diseases. In another embodiment, the therapeutic agents of the invention comprise genetic material such as nucleic acids (e.g., RNA, DNA, cDNA, etc.) that provide for gene therapy and/or virus-assisted gene transfer, such that a therapeutic protein can be synthesized within the cells of a treated subject or can be synthesized within cells that can then be used as a cell-based therapy for a subject.

Compounds and compositions of the invention can be delivered to a subject via any acceptable route including, but not limited to, intravenous, intraperitoneal, oral, mucosal, intramuscular, joint injection, intradermal, subcutaneous, intraosscous, transdermal, inhalation, etc. Compounds of the invention comprising lectin carrier and/or therapeutic agent can be produced in plant, fungal, mammalian or human cells or organisms. Compounds of the invention can also be produced using in vitro chemical synthesis means, including for example, chemical conjugation of lectin and therapeutic agent.

Examples of more common bone diseases include Osteoporosis, Osteopenia, Osteoarthritis, Osteomyelitis, Chondroblastoma, Chondrosarcoma, Osteosarcoma, Ewing's sarcoma, Multiple mieloma, Chondromyxoid Fibroma, Ankylosing Spondylitis, Rheumatoid Arthritis, Gout, Osteonecrosis, Fibromyalgia, Fibrous Dysplasia, Scoliosis, Paget's Disease of Bone, Osteopetrosis, Osteochondritis dissecans, Osteochondroma, Osteomalacia, Paget's disease of bone, and Rickets.

Examples of diseases for treatment within the scope of the subject invention include MPS IVA (Morquio A), MPS IVB (Morquio B), Glycogen Storage Disease type II (Pompe) and MPS I (Hurler syndrome). The therapeutic agent for use to treat MPS IVA comprises N-acetylgalactosamine-6-sulfatase (GALNS); the agent for MPS IVB comprises beta-galactosidase; the agent for Glycogen Storage Disease type II comprises acid alpha-glucosidase (GAA); the agent for MPS VI comprises arylsulfatase B (ARSB) and the agent for MPS I comprises Iduronidase (IDUA).

Examples of disease further includes, but not limited to, Muscular Dystrophy, Limb-Girdle, Autosomal Recessive 1; Glycogen Storage Disease 0, Muscle; Muscular Dystrophy, Limb-Girdle, Autosomal Recessive 21; Glycogen Storage Disease V; Glycogen Storage Disease Iii; Glycogen Storage Disease Iv; Glycogen Storage Disease Vi; Glycogen Storage Disease Vii; Glycogen Storage Disease Xii; Glycogen Storage Disease Type 0; Glycogen Storage Disease Xiii; Ehlers-Danlos Syndrome, Musculocontractural Type, 2; Ehlers-Danlos Syndrome, Musculocontractural Type, 1; Glycogen Storage Disease X; Glycogen Storage Dystrophy-Dystroglycanopathy; Muscular Dystrophy-Disease Xv; Muscular Dystroglycanopathy, Type B, 1; Autosomal Recessive Limb-Girdle Muscular Dystrophy Type 2a; Neuraminidase Deficiency; Niemann-Pick Disease, Type B; Mucopolysaccharidosis, Type Ii; Mucopolysaccharidosis, Type Iiib; Mucopolysaccharidosis, Type Iiia; Hurler Syndrome; Mucopolysaccharidosis, Type Iiid; Mucopolysaccharidosis, Type Iva; Mucopolysaccharidosis, Type Vi; Mucopolysaccharidosis, Type Ix; Mannosidosis, Alpha B, Lysosomal; Fucosidosis; Schindler Disease, Type I; Osteogenesis Imperfecta, Type Viii; Osteogenesis Imperfecta, Type Xi; Osteogenesis Imperfecta, Type Xiii; Osteogenesis Imperfecta, Type Xv; Osteogenesis Imperfecta, Type Xvii; Osteogenesis Imperfecta, Type Xx; Osteogenesis Imperfecta, Type Ix; Osteogenesis Imperfecta, Type Vii; Osteogenesis Imperfecta, Type X; Osteogenesis Imperfecta, Type Vi; and Osteogenesis Imperfecta, Type Xviii.

Examples of genetic diseases with skeletal manifestations within the scope of the invention include, but not limited to, osteogenesis imperfecta (OI). Therapeutic agents for use to treat OI include cartilage-associated protein (CRTAP) and prolyl 3-hydroxylase (P3H). In one embodiment, fusions of lectin and CRTAP, or lectin and P3H, can be prepared having a KDEL ER retrieval signal sequence.

In some embodiments, the system employs compounds comprising a plant lectin, such as the subunit B lectin proteins responsible for delivering toxin subunit A proteins to cells in the class of toxins known as AB protein exotoxins that utilize lectin binding sites on a targeted cell surface to gain entrance to cells via an endocytic pathway. AB toxins include the family of plant-made AB toxins (also called Toxalbumins), which include, but are not limited to, ricins, abrins, nigrins, the mistletoe lectins and the viscumin toxins, ebulins, pulchellin, pharatoxin, hurin, and phasin. These AB toxins are typically delivered to the cell via diverse endocytic pathways including the clathrin-dependent, clathrin-independent, and caveolae pathways.

In one embodiment of the present invention, a therapeutic protein or other compound is fused or linked to the subunit B, or a fragment or variant thereof, as a substitution for the natural toxic subunit A component. In some embodiments, the subunit B lectin protein is from ricin. In specific embodiments, the ricin B subunit that is utilized is truncated by removal of about 1 to 10 amino acids at the N-terminus of the protein. In an exemplified embodiment, the ricin B subunit is truncated wherein the first six amino acids of the protein are removed.

In specific embodiments, the fusion protein comprises a therapeutic protein fused to RTB. Preferably, the therapeutic protein is selected from IDUA, GALNS, GAA, CRTAP, P3H1, beta-Gal, and ARSB.

In specific embodiments, the fusion protein is IDUA fused to RTB, GALNS fused to RTB, GAA fused to RTB, CRTAP fused to RTB, P3H1 fused to RTB, BetaGal fused to RTB and ARSB fused to RTB.

This fusion protein may be produced by construction of a fusion gene incorporating a nucleotide sequence encoding a plant lectin (such as the subunit B lectin) and a nucleotide sequence encoding the therapeutic protein, and introducing this new genetic fusion (fusion gene) into a protein expression system, expressing the fusion protein encoded by the fusion gene, and isolating the fused protein for use as a therapeutic drug. Alternatively, the fusion may be accomplished by direct chemical fusion or conjugation yielding fusion of the plant lectin (such as a subunit B protein) with the therapeutic agent.

In one embodiment, the fusion protein comprises a linker or spacer sequence of amino acids between the plant lectin and the therapeutic protein or compound. Examples of linker or spacer sequences are well known in the art. Methods for preparing fusion genes and fusion protein are also well known in the art and have been described, for example, in U.S. Pat. Nos. 7,964,377; 7,867,972; 7,410,779; 7,011,972; 6,884,419; and 5,705,484.

In some embodiments, this linker peptide is a non-cleavable linker. In other embodiments, it is a non-cleavable rigid linker or non-cleavable flexible linker. In some embodiments, the linker peptide comprises 1 to 50 amino acids, 1 to 45 amino acids, 1 to 40 amino acids, 1 to 35 amino acids, 1 to 30 amino acids, 1 to 25 amino acids, 1 to 20 amino acids, 1 to 15 amino acids, 1 to 10 amino acids, 1 to 5 amino acids, 20 to 50 amino acids, 30 to 50 amino acids, 5 to 30 amino acids, 5 to 20 amino acids, 2 to 15 amino acids, 2 to 10 amino acids, or 3 to 5 amino acids. In specific embodiments, the linker peptide consists of 1 to 20 amino acids. For example, a rigid non-cleavable linker peptide can include 5 alanine amino acids.

In an additional embodiment of the invention, inter- and intracellular trafficking dynamics are directed or modified to enhance effectiveness as managed by sequence or chemical modifications of the fusion product of the invention. These modifications may include, for example, glycans, amino acids, nucleotides, peptides, and methylation. In still another embodiment, fusion products of the invention are produced in a stable or transient transgenic plant expression system. In one embodiment, a method for preparing a fusion product of the invention comprises expressing a polynucleotide encoding the fusion product in a cell and isolating the expressed fusion product from the cell.

Plant lectins that are contemplated within the scope of the invention include, but are not limited to, those B subunits from AB toxins such as ricins, abrins, nigrins, and mistletoe toxins, viscumin toxins, ebulins, pharatoxin, hurin, phasin, and pulchellin. They may also include lectins such as wheat germ agglutinin, peanut agglutinin, and tomato lectin that, while not part of the AB toxin class, are still capable of binding to animal cell surfaces and mediating endocytosis and transcytosis. Specific examples of plant lectins including their binding affinities and trafficking behavior are discussed further below.

Therapeutic compounds and agents contemplated within the scope of the invention include, but are not limited to, large molecular weight molecules including therapeutic proteins and peptides, siRNA, antisense oligonucleotides, and oligosaccharides. Other therapeutic compounds and agents contemplated within the scope of the invention include small molecular weight drug compounds including but not limited to vitamins, co-factors, effector molecules, and inducers of health promoting reactions. Examples of therapeutic compounds and agents are discussed further below.

Within the scope of the present invention, selection of a specific plant lectin candidate to use in delivery of a particular therapeutic compound or agent can be based on the specific sugar affinity of the lectin, its uptake efficiency into specific target cells, its pattern of intracellular trafficking, its in vivo biodistribution and pharmacodynamics, or other features of the lectin or therapeutic compound. Alternatively, multiple lectins can be tested to identify the lectin-therapeutic compound combination that provides greatest efficacy. For example, two lectins, RTB and NNB, were selected for proof-of-concept of the invention based on trafficking of their respective AB toxins, ricin from Ricinus communis and nigrin-b from Sambucus nigra (e.g., see Sandvig, K. and van Deurs, B. (1999); Simmons et al. (1986); Citores et al. (1999); Citores et al. (2003)).

The uptake and trafficking of ricin and/or RTB, a galactose/galactosamine-specific lectin, has been extensively studied. This lectin has high affinity for surface glycolipids and glycoproteins providing access to a broad array of cells and enters cells by multiple endocytic routes. These include absorptive-mediated endocytosis involving clathrin-dependent and clathrin-independent routes; caveolin-dependent and independent routes; dynamin-dependent and independent routes, and macropinocytosis based on the lectin binding to cell surface glycoproteins and glycolipids. RTB also accesses cells by receptor-mediated endocytosis based on interaction with its N-linked glycans with the high-mannose receptor (MMR) of animal cells. Upon endocytosis, RTB traverses preferentially to lysosomes (lysosomal pathway) or cycles back to the cell membrane (transcytosis pathway), with a small amount (generally less than 5%) moving “retrograde” to the endoplasmic reticulum.

The NBB lectin, Nigrin B B-subunit from Sambucus nigra, exploits different uptake and intracellular trafficking routes compared to RTB, and thus provides unique in vivo pharmacodynamics. In contrast to RTB, NBB has strong affinity for N-acetyl-galactosamine, low affinity for lactose, very limited retrograde trafficking but strong accumulation in lysosomes. Plant-made DsReD:NNB (red fluorescent protein-NBB fusion) is rapidly taken up into multiple mammalian cells and efficiently delivered to lysosomes.

As delineated further in the Examples, recombinantly produced RTB and NBB have been operatively associated with both small molecules (by chemical conjugation technologies) and protein macromolecule by genetic fusion that retain selective lectin binding as well as functionality of the associated protein or agent. These operatively associated products are rapidly endocytosed into multiple cell types and tissues and deliver fully functional ‘payload’ into internal structures including lysosomes, endosomes, endoplasmic reticulum, and sarcoplasmic reticulum. Of particular significance, these lectins mobilize delivery of enzymes and other large proteins into critical cells of the central nervous system (including, but not limited to, brain capillary endothelial cells, neurons, and astrocytes), skeletal systems (including but not limited to cartilage, chondrocytes, fibroblasts, and monocytes), and the respiratory system (including but not limited to lung airway epithelium, lung smooth muscle cells, and macrophages). These cells and tissues represent some of the most challenging targets for delivery of therapeutic agents highlighting the utility and novelty of the current invention to address currently unmet needs in therapeutic compound delivery in human and animal medicine.

Additional plant lectins that are contemplated within the scope of the invention are those having particular carbohydrate-binding affinities including but not limited to lectins that bind glucose, glucosamine, galactose, galactosamine, N-acetyl-glucosamine, N-acetyl-galactosamine, mannose, fucose, sialic acid, neuraminic acid, and/or N-acetylneuraminic acid, or have high affinity for certain target tissue or cells of interest. There are hundreds of plant lectins that have been identified and experimental strategies to identify plant lectins, their respective genes, and their sugar binding affinities are widely known by those skilled in the art. The diversity of plant sources for lectins and their sugar binding affinities is exemplified in the Table below (adapted from Table 3 of Van Damme et al., (1998)).

Type 2 Ribosome-Inactivating Proteins and Related Lectins:
Occurrence, Molecular Structure, and Specificity

| | | | | Sequence |
|---|---|---|---|---|
| Species | Tissue | Structure^{a} | Specificity | Available^{b} |
| Marolectins | | | | |
| Sambucus nigra | Bark | [P22] | NANA | Nu |
| | Fruit | [P22] | NANA | Nu |
| Holoectins | | | | |
| Sambucus nigra | Bark | II [P30]₂ | GalNAc > Gal | Nu |
| | Seed | III [P30]₂ | GalNAc > Gal | |
| | Fruit | IVf [P32]₂ | Gal/GalNAc | Nu (SNA-IV) |
| | Leaf | IVI [P32]₂ | Gal/GalNAc | Nu |
| | Leaf | IV4I [P32]₄ | Gal/GalNAc | |
| Chimerolectins | | | | |
| Abrus precatorius | Seed | [P(34 + 32)] | Gal > GalNAc | Pr, Nu (Abrin) |
| | Seed | [P (33 + 29)]₂ | Gal | Pr (APA) |
| Adenia digitata | Root | [P(28 + 38)] | Gal > GalNAc | |
| Adenia volkensii | Root | [P(29 + 36)] | Gal | |
| Cinnamonum camphora | Seed | [P(30 + 33)]_{?} | Unknown | |
| Eranthis hyemalis | Tuber | [P(30 + 32)] | GalNAc | |
| Iris hybrid | Bulb | [P(27 + 34)] | GalNAc | |
| Momordica charantia | Seed | [P(28 + 30)]₂ | Gal > GalNAc | |
| Phoradendron californicum | Plant | [P(31 + 38)] | Gal | |
| Ricinus communis | Seed | [P(32 + 34)] | Gal > GalNAc | Pr, Nu (Ricin) |
| | Seed | [P(32 + 36)]₂ | Gal >> GalNAc | Pr, Nu (RCA) |
| Sambucus canadensis | Bark | I [P(32 + 35)]₄ | NANA | |
| Sambucus ebulus | Bark | I [P(32 + 37)]₄ | NANA | |
| | Leaf | [P(26 + 30)]₂ | GalNAc | |
| Sambucus nigra | Seed | Vs [P(26 + 32)]₂ | GalNAc > Gal | |
| | Bark | I [P(32 + 35)]₄ | NANA | Nu (SNA-I) |
| | Bark | I′ [P(32 + 35)₂ | NANA | Nu (SNA-I′) |
| | Bark | V [P(26 + 32)]₂ | GalNAc > Gal | Nu (SNA-V) |
| | Fruit | If [P(32 + 35)]₂ | NANA | Nu |
| | Fruit | Vf [P(26 + 32)]₂ | GalNAc > Gal | Nu |
| Sambucus racemosa | Bark | I [P(30 +36)]₄ | NANA | |
| Sambucus sieboldiana | Bark | I [P(31 + 37)]₄ | NANA | Nu (SSA-I) |
| | Bark | [P(27 + 32)] | GalNAc > Gal | Nu (Sieboldin) |
| Viscum album | Plant | I [P(29 + 34 )]₁₋₂ | Gal | |
| | Plant | II[P(29 + 34 )] | Gal/GalNAc | |
| | Plant | III [P(25 + 30)] | GalNAc > Gal | |
| Type 2 RIP with inactive B chain | | | | |
| Sambucus nigra | Bark | [P(32 + 32)] | — | Nu (LRPSN) |

^{a}[PX] stands for protomer with a molecular mass of X kDa. [P(Y + Z)] Indicates that the protomer is cleaved in two polypeptides of Y and Z kDa.
^{b}Pr, protein sequence; Nu, nucleotide sequence. The abbreviation in brackets refers to the sequence name used in the dendrogram (FIG. 20).

As a further example of plant lectins contemplated herein, the Table below exemplifies the large number of different lectins identified from the Sambucus species alone. This group includes nigrin B, the source on NBB.

Ribosome-inactivating proteins (RIPs) and lectins from Sambucus
species. Adapted from Table 1 of Ferreras et al. (2011)

| Proteins | Species | Tissues |
|---|---|---|
| Type 1 RIPs | | |
| Ebulitins α, β and γ | S. ebulus | Leaves |
| Nigritins f1 and f2 | S. nigra | Fruits |
| Heterodimeric type 2 RIPs | | |
| Ebulin 1 | S. ebulus | Leaves |
| Ebulin f | S. ebulus | Fruits |
| Ebulins r1 and r2 | S. ebulus | Rhizome |
| Nigrin b, basic nigrin b, SNA I′, SNLRPs | S. nigra | Bark |
| Nigrins 11 and 12 | S. nigra | Leaves |
| Nigrin f | S. nigra | Fruits |
| Nigrin s | S. nigra | Seeds |
| Sieboldin b | S. sieboldiana | Bark |
| Basic racemosin b | S. racemosa | Bark |
| Tetrameric type 2 RIPs | | |
| SEA | S. ebulus | Rhizome |
| SNA I | S. nigra | Bark |
| SNAIf | S. nigra | Fruits |
| SNAflu-I | S. nigra | Flowers |
| SSA | S. sieboldiana | Bark |
| SRA | S. racemosa | Bark |
| Monomeric lectins | | |
| SEL1m | S. ebulus | Leaves |
| SEA II | S. ebulus | Rhizome |
| SNA II | S. nigra | Bark |
| SNA1m and SNAIV1 | S. nigra | Leaves |
| SNA IV | S. nigra | Fruits |
| SNA III | S. nigra | Seeds |
| SSA-b-3 and SSA-b-4 | S. sieboldiana | Bark |
| SRAbm | S. racemosa | Bark |
| Homodimeric lectins | | |
| SEL1d | S. ebulus | Leaves |
| SELfd | S. ebulus | Fruits |
| SNA1d | S. nigra | Leaves |

The subject invention also provides polynucleotides that comprise nucleotide sequences encoding a fusion protein of the invention. In one embodiment, the polynucleotides comprise nucleotide sequences that are optimized for expression in a particular expression system, e.g., a plant expression system, such as a tobacco plant. The subject invention also provides the fusion polypeptides encoded by polynucleotides of the invention.

Any disease or disorder that can be treated or prevented using a therapeutic compound or agent is contemplated within the scope of the present invention. In one embodiment, the disease or disorder is one of the musculoskeletal systems including bone, muscle, cartilage, and/or connective tissue. Diseases of the central nervous system can also be treated.

Lysosomal diseases and related enzymes and proteins associated with diseases that are contemplated within the scope of the invention include, but are not limited to, Activator Deficiency/GM2 Gangliosidosis (beta-hexosaminidase), Alpha-mannosidosis (alpha-D-mannosidase), Aspartylglucosaminuria (aspartylglucosaminidase), Cholesteryl ester storage disease (lysosomal acid lipase), Chronic Hexosaminidase A Deficiency (hexosaminidase A), Cystinosis (cystinosin), Danon disease (LAMP2), Fabry disease (alpha-galactosidase A), Farber disease (ceramidase), Fucosidosis (alpha-L-fucosidase), Galactosialidosis (cathepsin A), Gaucher Disease (Type I, Type II, Type III) (beta-glucocerebrosidase), GM1 gangliosidosis (Infantile, Late infantile/Juvenile, Adult/Chronic) (beta-galactosidase), I-Cell disease/Mucolipidosis II (GlcNAc-phosphotransferase), Infantile Free Sialic Acid Storage Disease/ISSD (sialin), Juvenile Hexosaminidase A Deficiency ((hexosaminidase A), Krabbe disease (Infantile Onset, Late Onset) (galactocerebrosidase), Metachromatic Leukodystrophy (arylsulfatase A), Mucopolysaccharidoses disorders [Pseudo-Hurler polydystrophy/Mucolipidosis IIIA (N-acetylglucosamine-1-phosphotransferase), MPSI Hurler Syndrome (alpha-L iduronidase), MPSI Scheie Syndrome (alpha-L iduronidase), MPS I Hurler-Scheie Syndrome (alpha-L iduronidase), MPS II Hunter syndrome (iduronate-2-sulfatasc), Sanfilippo syndrome Type A/MPS III A (heparan N-sulfatase), Sanfilippo syndrome Type B/MPS III B (N-acetyl-alpha-D-glucosaminidase), Sanfilippo syndrome Type C/MPS III C (acetyl-CoA, alpha-glucosaminide acetyltransferase, Sanfilippo syndrome Type D/MPS III D (N-acetylglucosamine-G-sulfate-sulfatase), Morquio Type A/MPS IVA (N-acetylgalatosamine-6-sulfate-sulfatase), Morquio Type B/MPS IVB (β-galactosidase-1), MPS IX Hyaluronidase Deficiency (hyaluronidase), MPS VI Maroteaux-Lamy (arylsulfatase B), MPS VII Sly Syndrome (beta-glucuronidase), Mucolipidosis I/Sialidosis (alpha-N-acetyl neuraminidase), Mucolipidosis IIIC (N-acetylglucosamine-1-phosphotransferase), Mucolipidosis type IV (mucolipin1)], Multiple sulfatase deficiency (multiple sulfatase enzymes), Niemann-Pick Disease (Type A, Type B, Type C) (sphingomyelinase), Neuronal Ceroid Lipofuscinoses [(CLN6 disease-Atypical Late Infantile, Late Onset variant, Early Juvenile (ceroid-lipofuscinosis neuronal protein 6); Batten-Spielmeyer-Vogt/Juvenile NCL/CLN3 disease (battenin); Finnish Variant Late Infantile CLN5 (ceroid-lipofuscinosis neuronal protein 5); Jansky-Bielschowsky disease/Late infantile CLN2/TPP1 Disease (tripeptidyl peptidase 1); Kufs/Adult-onset NCL/CLN4 disease; Northern Epilepsy/variant late infantile CLN8 (ceroid-lipofuscinosis neuronal protein 8); Santavuori-Haltia/Infantile CLN1/PPT disease (palmitoyl-protein thioesterase 1); Beta-mannosidosis (beta-mannosidase)], Tangier disease (ATP-binding cassette transporter ABCA1), Pompe disease/Glycogen storage disease type II (acid maltase), Pycnodysostosis (cathepsin K), Sandhoff disease/Adult Onset/GM2 Gangliosidosis (beta-hexosaminidases A and B), Sandhoff disease/GM2 gangliosidosis-Infantile, Sandhoff disease/GM2 gangliosidosis-Juvenile (beta-hexosaminidases A and B), Schindler disease (alpha-N-acetylgalactosaminidas), Salla disease/Sialic Acid Storage Disease (sialin), Tay-Sachs/GM2 gangliosidosis (beta-hexosaminidase), and Wolman disease (lysosomal acid lipase), Sphingolipidosis, Hurmansky-Pudiak Syndrome (HPS1, HPS3, HPS4, HPS5, HPS6 and HPS7) Type 2-AP-3 complex subunit beta-1, Type 7-dysbindin), Chediak-Higashi Syndrome (lysosomal trafficking regulator protein), and Griscelli disease (Type 1: myosin-Va, Type 2: ras-related protein Rab-27A, Type 3: melanophilin).

ERT's or other protein replacement therapeutics may be of value for these diseases. Lectins may facilitate protein delivery to critical organs, cells and subcellular organelles or compartments for these diseases as well. For example, genetic diseases affecting bone and connective tissues including, but are not limited to osteoporosis and osteogenesis imperfecta, may be treated by using this invention to deliver corrective proteins to bones, joints, and other connective tissues.

Additional muscle and bone diseases and genes involved can be found in the Table below:

| | | Type of | | |
|---|---|---|---|---|
| | | protein | | |
| Disease | Gene | encoded | | Localization |
| Muscular Dystrophy, Limb-Girdle, Autosomal | CAPN3 | Protease | Calcium-regulated non-lysosomel thiol-protease. | Cytosol |
| Recessive 1 | | | | |
| Glycogen Storage Disease 0, Muscle | GYS1 | Enzyme | Glycogen Synthase 1 | Cytosol |
| Muscular Dystrophy, Limb-Girdle, Autosomal | POGLUT1 | Enzyme | O-glucosyltransferase and O-xylosyltransferase | ER lumen |
| Recessive 21 | | | | |
| Glycogen Storage Disease Ii | GAA | Enzyme | degradation of glycogen to glucose in lysosomes | Lysosome |
| Glycogen Storage Disease V | PYGM | Enzyme | Glycogen Phosphorylase, Muscle Associated | Cytosol |
| Glycogen Storage Disease Iii | AGL | Enzyme | glycogen debrancher enzyme | Cytosol |
| Glycogen Storage Disease Iv | GBE1 | Enzyme | glycogen branching enzyme | Cytosol |
| Glycogen Storage Disease Vi | PYGL | Enzyme | cleavage of alpha-1,4-glucosidic bonds | Cytosol |
| Glycogen Storage Disease Vii | PFKM | Enzyme | phosphorylation of D-fructose 6-phosphate | Cytosol |
| Glycogen Storage Disease Xii | ALDOA | Enzyme | fructose-bisphosphate aldolase | Cytosol |
| Glycogen Storage Disease Type 0 | GYS2 | Enzyme | catalyzes the rate-limiting step in the synthesis of | Cytosol |
| | | | glycogen | |
| Glycogen Storage Disease Xiii | ENO3 | Enzyme | muscle development and regeneration | Cytosol |
| Ehlers-Danlos Syndrome, Musculocontractural | DSE | Enzyme | convert D-glucuronic acid to L-iduronic acid during | ER membrane |
| Type, 2 | | | the biosynthesis of dermatan sulfate | |
| Ehlers-Danlos Syndrome, Musculocontractural | CHST14 | Enzyme | transfers sulfate to the C-4 hydroxyl of N- | Golgi membrane |
| Type, 1 | | | acetylgalactosamine residues in dermatan sulfate | |
| Glycogen Storage Disease X | PGAM2 | Enzyme | 3-phosphoglycerate (3-PGA) to 2-phosphoglycerate | Cytosol |
| | | | (2-PGA) in the glycolytic pathway. | |
| Glycogen Storage Disease Xv | GYG1 | Enzyme | glycosyltransferase that catalyzes the formation of a | Lysosome |
| | | | short glucose polymer | |
| Muscular Dystrophy-Dystroglycanopathy | FKRP | Enzyme | posttranslational modification of dystroglycan | Golgi membrane |
| Muscular Dystrophy-Dystroglycanopathy, Type | POMT1 | Enzyme | O-mannosyltransferase | ER Membrane |
| B, 1 | | | | |
| Autosomal Recessive Limb-Girdle Mascular | CAPN3 | Enzyme | intracellular protease | Cytosol |
| Dystrophy Type 2a | | | | |
| Neuraminidase Deficiency | NEU1 | Enzyme | Neuraminidase | Lysosome |
| Niemann-Pick Disease, Type B | SMPD1 | Enzyme | Niemann-Pick disease type A (NPA) and Niemann- | Lysosome |
| | | | Pick disease type B (NPB) | |
| Mucopolysaccharidosis, Type Ii | IDS | Enzyme | hunter syndrome | Lysosome |
| Mucopolysaccharidosis, Type Iiib | NAGLU | Enzyme | Sanfilippo Syndrome Type B | Lysosome |
| Mucopolysaccharidosis, Type Iiia | SGSH | Enzyme | Sanfilippo Syndrome a | Lysosome |
| Hurler Syndrome | IDUA | Enzyme | MPSI | Lysosome |
| Mucopolysaccharidosis, Type Iiid | GNS | Enzyme | Sanfilippo Disease IIID | Lysosome |
| Mucopolysaccharidosis, Type Iva | GALNS | Enzyme | Morquio A | Lysosome |
| Mucopolysaccharidosis, Type Vi | ARSB | Enzyme | | Lysosome |
| Mucopolysaccharidosis, Type Ix | HYAL1 | Enzyme | Hyaluronidase Deficiency | Lysosome |
| Mannosidosis, Alpha B, Lysosomal | MAN2B1 | Enzyme | | Lysosome |
| Fucosidosis | FUCA1 | Enzyme | | Lysosome |
| Schindler Disease, Type I | NAGA | Enzyme | Alpha-N-Acetylgalactosaminidase | Lysosome |
| Osteogenesis Imperfecta, Type Viii | P3H1 | | | ER |
| Osteogenesis Imperfecta, Type Xi | FKBP10 | chaperone | | ER |
| Osteogenesis Imperfecta, Type Xiii | BMP1 | Growth Factor | inducing formation of cartilage | Golgi |
| Osteogenesis Imperfecta, Type Xv | WNT1 | Growth Factor | | ER |
| Osteogenesis Imperfecta, Type Xvii | SPARC | Growth Factor | required for the collagen in bone to become calcified | ER |
| Osteogenesis Imperfecta, Type Xx | MESD | Receptor | Involved in ossification and protein folding. | ER |
| | | binding | | |
| Osteogenesis Imperfecta, Type Ix | PPIB | | regulate cyclosporine A-mediated | ER |
| | | | immunosuppression | |
| Osteogenesis Imperfecta, Type Vii | CRTAP | scaffolding | may influence the activity of at least one member of | ER |
| | | | the cytohesin/ARNO family | |
| Osteogenesis Imperfecta, Type X | SERPINH1 | sezine | plays a role in collagen biosynthesis as a collagen- | |
| | | proteinase | specific molecular chaperone. | |
| | | inhibitors | | |
| Osteogenesis Imperfecta, Type Vi | SERPINF1 | serine | plays a role in collagen biosynthesis as a collagen- | Extracellular |
| | | proteinase | specific molecular chaperone. | |
| | | inhibitors | | |
| Osteogenesis Imperfecta, Type Xviii | TENT5A | RNA binding | Predicted to be involved in mRNA stabilization. | Cytosol |

In some embodiments, the disease may be caused by genetic mutations in genes such as CAPN3, GYS1, POGLUT1, GAA, PYGM, AGL, GBE1, PYGL, PFKM, ALDOA, GYS2, ENO3, DSE, CHST14, PGAM2, GTG1, FKRP, POMT1, CAPN3, NEU1, SMPD1, IDS, NAGLU, SGSH, IDUA, GNS, GALNS, ARSB, HYAL1, MAN2B1, FUCA1, NAGA, P3H1, FKBP10, BMP1, WNT1, SPARC, MESD, CRTAP, SERPINH1, SERPINF1 and TENT5A.

In one embodiment, the clinical manifestations in muscle may be caused by genetic mutations in genes such as CAPN3, GYS1, POGLUT1, GAA, PYGM, AGL, GBE1, PYGL, PFKM, ALDOA, GYS2, ENO3, DSE, CHST14, PGAM2, GTG1, FKRP, POMT1, CAPN3, NEU1. In one embodiment, the clinical manifestations in joints may be caused by genetic mutations in genes such as ASAHI, GLB1, IDUA, IDS, SGSH, NAGLU, HGSNAT, GNS, GALNS, and ARSB. In one embodiment, the clinical manifestations in skeletal tissue may be caused by genetic mutations in genes such as GLB1, DBA, SMPD1, IDUA, IDS, SGSH, NAGLU, HGSNAT, GNS, GALNS, ARSB, HYAL1, MAN2B1, FUCA1, NAGA, CTSA, GNPTAB, GNPTAG, CTNS, P3H1, FKBP10, BMP1, WNT1, SPARC, MESD, CRTAP, SERPINH1, SERPINF1, TENT5A and NEU1.

In one embodiment, the disease (and associated gene) may be selected from:
1) Sphingolipidoses: Fabry disease (GLA), Farber lipogranulomatosis (ASAHI), Gaucher disease: type I, type II, type III and perinatal lethal form (GBA), GM1 gangliosidosis: type I, type II and type III (GLB1), GM2 gangliosidosis, Tay-Sachs disease (HEXA), GM2 gangliosidosis, Sandhoff disease (HEXB), GM2 gangliosidosis, GM2 activator deficiency (GM2A), Globoid cell leukodystrophy, also known as Krabbe disease (GALC), Metachromatic leukodystrophy (ARSA and PSAP), Niemann-Pick disease types A and B (SMPD1);
2) Mucopolysaccharidoses: MPS I: Hurler syndrome, Hurler-Scheie syndrome and Scheie syndrome (IDUA), MPS II, also known as Hunter syndrome (IDS), MPS IIIA, also known as Sanfilippo syndrome A (SGSH), MPS IIIB, also known as Sanfilippo syndrome B (NAGLU), MPS IIIC, also known as Sanfilippo syndrome C (HGSNAT), MPS IIID, also known as Sanfilippo syndrome D (GNS), MPS IVA, also known as Morquio syndrome A (GALNS), MPS IVB, also known as Morquio syndrome B (GLB1), MPS VI, also known as Maroteaux-Lamy syndrome (ARSB), MPS VII, also known as Sly disease (GUSB), MPS IX (HYAL1);
3) Glycogen storage disease (GSD): GSD II, also known as Pompe disease (GAA);
4) Glycoproteinoses: α-Mannosidosis: type I mild, type II moderate and type III severe (MAN2B1), β-Mannosidosis (MANBA), Fucosidosis (FUCA1), Aspartylglucosaminuria (AGA), Schindler disease: type I, also known as infantile-onset neuroaxonal dystrophy, type II also known as Kanzaki disease, and type III, intermediate severity (NAGA), Sialidosis type I, also known as cherry-red spot myoclonus syndrome (NEU1), Sialidosis type II, also known as mucolipidosis I (NEU1), Galactosialidosis (CTSA), Lipid storage diseases, Acid lipase deficiency: Wolman disease and cholesterol ester storage disease (LIPA);
5) Post-translational modification defects: Multiple sulfatase deficiency (SUMF1), Mucolipidosis II α/β, I-cell disease (GNPTAB), Mucolipodosis II α/β, pseudo-Hurler polydystrophy (GNPTAB), Mucolipidosis III Y, variant pseudo-Hurler polydystrophy (GNPTG);
6) Integral membrane protein disorders: Cystinosis (CTNS), Danon disease (LAMP2), Action myoclonus-renal failure syndrome (SCARB2), Sialic acid storage disease: ISSDb, Salla disease and intermediate severity Salla disease (SLC17A5), Niemann-Pick disease types C1 and C2 (NPC1 and NPC2), Mucolipidosis IV (MCOLN1)
7) Neuronal ceroid lipofuscinoses: Neuronal ceroid lipofuscinoses CLN1: Haltia-Santavuori disease and INCL (PPT1), CLN2, also known as Jansky-Bielschowsky disease (TPP1), CLN3, also known as Batten-Spielmeyer-Sjogren disease (CLN3), CLN4: Parry disease and Kufs type A and B (DNAJC5), CLN5: Finnish variant late infantile (CLN5), CLN6: Lake-Cavanagh or Indian variant (CLN6), CLN7: Turkish variant (MFSD8), CLN8: northern epilepsy, epilepsy mental retardation (CLN8), CLN9 (N/A), CLN10 (CTSD), CLN11 (GRN), CLN12: Kufor-Rakeb syndrome or PARK9 (ATP13A2), CLN13 (CTSF), CLN14 (KCTD7); and
8) LRO disorders: Hermansky-Pudlak disease type 1 (HPS1), Hermansky-Pudlak disease type 2 (HPS2), Hermansky-Pudlak disease type 3 (HPS3), Hermansky-Pudlak disease type 4 (HPS4), Hermansky-Pudlak disease type 5 (HPS5), Hermansky-Pudlak disease type 6 (HPS6), Hermansky-Pudlak disease type 7 (HPS7), Hermansky-Pudlak disease type 8 (HPS8), Hermansky-Pudlak disease type 9 (HPS9), Griscelli syndrome 1, also known as Elejalde syndrome (MYO5A), Griscelli syndrome 2 (RAB27A), Chédiak-Higashi disease (LYST).

Many other genetic diseases are caused by deficiencies in specific proteins or enzymes leading to disease specific tissue and organ pathologies.

The enzymes or other proteins that can be used therapeutically in a fusion protein of the present invention can be identified by a person of ordinary skill in the art. For example, in treating MPS I disease, the therapeutic protein can provide iduronidase enzymatic activity. For treating Fabry disease, the therapeutic protein can provide α-galactosidase A enzymatic activity. Enzymes suitable for treating other LSDs are known in the art.

The present invention contemplates products in which the plant lectin is operatively associated with the therapeutic component by one of many methods known in the art. For example, genetic fusions between a plant lectin protein and a therapeutic protein can orient the lectin partner on either the C- or N-terminus of the therapeutic component. The coding regions can be linked precisely such that the last C-terminal residue of one protein is adjacent to the first N-terminal residue of the mature (i.e., without signal peptide sequences) second protein. Alternatively, additional amino acid residues can be inserted between the two proteins as a consequence of restriction enzyme sites used to facilitate cloning at the DNA level. Additionally, the fusions can be constructed to have amino acid linkers between the proteins to alter the physical spacing. These linkers can be short or long, flexible (e.g., the commonly used (Gly₄Ser)₃ ‘flexi’ linker) or rigid (e.g., alpha helix or containing spaced prolines), provide a cleavage domain (e.g., see Chen et al. (2010)), or provide cysteines to support disulfide bond formation.

The plant lectins are glycoproteins and in nature are directed through the plant endomembrane system during protein synthesis and post-translational processing. For this reason, production of recombinant fusion proteins comprising a plant lectin and a therapeutic protein partner may require that a signal peptide be present on the N-terminus of the fusion product (either on the lectin or on the therapeutic protein depending on the orientation of the fusion construct) in order to direct the protein into the endoplasmic reticulum during synthesis. This signal peptide can be of plant or animal origin and is typically cleaved from the mature plant lectin or fusion protein product during synthesis and processing in the plant or other cukaryotic cell. In one embodiment, a modified patatin signal sequence (PoSP) is utilized: MASSATTKSFLILFFMILATTSSTCAVD (SEQ ID NO: 1) (see GenBank accession number CAA27588.1, version GI:21514 by Bevan et al. and referenced at “The structure and transcription start site of a major potato tuber protein gene” Nucleic Acid Res. 14 (11), 4625-4638 (1986)). Other signal peptides can be used to increase secretion of products to extracellular space in mammalian cell systems (recombinant or gene therapy). Examples of these includes albumin (MKWVTFISLLFLFSSAYS; SEQ ID NO: 2), Azurocidin (MTRLTVLALLAGLLASSRA; SEQ ID NO: 3), Cathepsin Z (MASSGSVQQPRLVLLMLVLAGAARA; SEQ ID NO: 4), Metalloproteinase inhibitor 1 (MAPFASLASGILLLLSLITSSKA; SEQ ID NO: 5).

Compounds of the subject invention can also be prepared by producing the plant lectin and the therapeutic drug or protein separately and operatively linking them by a variety of chemical methods. Examples of such in vitro operative associations include conjugation, covalent binding, protein-protein interactions or the like (see, e.g., Lungwitz et al. (2005); Lovrinovic and Niemeyer (2005)). For example, N-hydroxysuccinimde (NHS)-derivatized small molecules and proteins can be attached to recombinant plant lectins by covalent interactions with primary amines (N-terminus and lysine residues). This chemistry can also be used with NHS-biotin to attach biotin molecules to the plant lectin supporting subsequent association with streptavidin (which binds strongly to biotin) and which itself can be modified to carry additional payload(s). In another example, hydrazine-derivatized small molecules or proteins can be covalently bound to oxidized glycans present on the N-linked glycans of the plant lectin.

Proteins can also be operatively linked by bonding through intermolecular disulfide bond formation between a cysteine residue on the plant lectins and a cysteine residue on the selected therapeutic protein. It should be noted that the plant AB toxins typically have a single disulfide bond that forms between the A and B subunits. Recombinant production of plant B subunit lectins such as RTB and NBB yield a product with an ‘unpaired’ cysteine residue that is available for disulfide bonding with a “payload” protein. Alternatively, this cysteine (e.g., Cys4 in RTB) can be eliminated in the recombinant plant lectin product by replacement with a different amino acid or elimination of the first 4-6 amino acids of the N-terminus to eliminate the potential for disulfide bonding with itself or other proteins.

Plant lectin fusion proteins of the present invention include genetic fusions wherein a) the plant lectin is either RTB (the B subunit of ricin) or NBB (the B subunit of nigrin B), b) the associated protein is either human GALNS, human β-galactosidase, or human ARSB, c) the plant lectin comprises either the C-terminal or N-terminal partner of the fusion protein, d) the fusion protein comprises a precise fusion between partners adding no additional amino acids, e) the fusion protein includes at least one or two additional amino acids resulting from an added restriction site in cloning, and/or f) modifications are made at the C- or N-termini of one or both protein partners to add or eliminate a signal peptide, remove a cysteine residue, or add a C-terminal histidine tag. All fusion products are tested for functionality of both partners (e.g., plant lectin carbohydrate binding selectivity and enzyme activity or fluorescence of its fusion partner) and optimal activity of one or both partners can be affected by the fusion arrangement (N-versus C-terminal) and physical spacing.

Additional information concerning IDUA, SGSH, NBB, GALNS, GAA and RTB can be found at:

IDUA: See GenBank accession number pbd/4JXP_A version GI:480312357 by Bie et al. and referenced at “Crystal structure analysis of human alpha-L-iduronidase two crystal forms” Unpublished, and see GenBank accession number AAA81589.1, version GI: 184559 by Scott et al. and referenced at “Human alpha-L-iduronidase: cDNA isolation and expression” Proc. Natl. Acad. Sci. U.S.A. 88 (21), 9695-9699 (1991).

NBB: See GenBank accession number P33183.2, version GI:17433713 (containing subunits A and B) by Van Damme et al. and referenced at “Characterization and molecular cloning of Sambucus nigra agglutinin V (nigrin b), a GalNAc-specific type-2 ribosome-inactivating protein from the bark of elderberry (Sambucus nigra)” Eur. J. Biochem. 237 (2), 505-513 (1996). PDB ID: 3CA3 (for B subunit) by Maveyraud et al. and referenced at “Structural basis for sugar recognition, including the tn carcinoma antigen, by the lectin sna-ii from sambucus nigra” Proteins 75 p. 89 (2009).

SGSH: See GenBank accession number NP_000190.1, version GI:4506919 by Van de Kamp et al. and referenced at “Genetic heterogeneity and clinical variability in the Sanfilippo syndrome (type A, B, and C)” Clin. Genet. 20 (2), 152-160 (1981).

GALNS: See GenBank accession number NG_008667 by Regier et al. and referenced at “Mucopolysaccharidosis Type IVA” genereviews (1993).

GAA: See GenBank accession number NC_000017 by Zody, M. C. et al. and referenced at “DNA sequence of human chromosome 17 and analysis of rearrangement in the human lineage” Nature 440 (7087), 1045-1049 (2006).

RTB: See GenBank accession number pbd/2AAI/B, version GI:494727 (containing subunits A and B) by Montfort et al. and referenced at “The three-dimensional structure of ricin at 2.8A” J. Biol Chem. 262 (11), 5398-5403 (1987).

In vivo administration of the subject compounds, polynucleotides and compositions containing them, can be accomplished by any suitable method and technique presently or prospectively known to those skilled in the art. The subject compounds can be formulated in a physiologically or pharmaceutically acceptable form and administered by any suitable route known in the art including, for example, oral, nasal, rectal, transdermal, vaginal, and parenteral routes of administration. As used herein, the term parenteral includes subcutaneous, intradermal, intravenous, intramuscular, intraperitoneal, and intrasternal administration, such as by injection. Administration of the subject compounds or composition comprising the compounds of the invention can be a single administration, or at continuous or distinct intervals as can be readily determined by a person skilled in the art.

In one embodiment, a polynucleotide encoding a therapeutic fusion product of the invention is stably incorporated into the genome of a person of animal in need of treatment. Methods for providing gene therapy are well known in the art including viral and non-viral methods. For example, AAV is the most common viral vector for delivering gene therapy, accounting for almost half of all viral vector-based gene therapies currently in clinical trials and three of the four viral vector gene therapies that have been approved by the FDA to date. Recombinant AAVs (rAAVs) are well suited for treating disorders because they stably transfect cells to support the continuous production of the encoded protein.

In one embodiment, the subject invention provides the use of gene therapy for in situ production of a therapeutic fusion product, e.g., a lectin-based fusion protein, to treat, for example, the diseases or disorders disclosed herein. In a specific embodiment, the gene therapy is an AAV-based gene therapy.

In some embodiments, the gene therapy involves the administration of a polynucleotide sequence encoding a therapeutic fusion product of the subject invention. In certain embodiments, the polynucleotide sequence encoding the fusion product is provided in an expression construct such as a virus vector, e.g., AAV vector or a non-viral vector. A suitable vector may be any vector that is capable of carrying a sufficient amount of genetic information and allowing expression of a fusion product in vivo. A vector comprising a nucleic acid construct of the invention may be administered directly to a patient in need thereof. Such vectors are routinely constructed in the art of molecular biology and may for example involve the use of plasmid DNA and appropriate initiators, promoters, enhancers and other elements, such as for example polyadenylation signals, in order to allow for expression of a peptide of the invention. Other suitable vectors would be apparent to persons skilled in the art, as described by way of further example, in Sambrook et al. (1989, Molecular Cloning—a laboratory manual; Cold Spring Harbor Press). The gene therapy vector can be constructed and cloned by standard methods known in the art, such as recombinant DNA technology or chemical synthesis. Standard cloning methods are described e.g., in Sambrook et al. (1989, Molecular Cloning—a laboratory manual; Cold Spring Harbor Press).

In one embodiment, the vector should be specifically adapted to provide expression of the encoded protein in a cell. In a preferred embodiment, the vector provides specific expression of the encoded protein in liver cells or musculoskeletal cells. For example, the gene therapy vector may be part of a mammalian expression system. Useful mammalian expression systems and expression constructs have been described in the prior art. Also, several mammalian expression systems are distributed by different manufacturers and can be employed in the present invention, such as plasmid- or viral vector-based systems, e.g., LENTI-Smart™, GenScript™ Expression vectors, pAdVAntage™, ViraPower™ Lentiviral, and Adenoviral Expression Systems.

In certain embodiments, the expression vector can be an episomal vector, i.e., one that is capable of self-replicating autonomously within the host cell, or an integrating vector, i.e., one that stably incorporates into the genome of the cell. The expression in the host cell can be constitutive or regulated (e.g., inducible).

In preferred embodiments, the gene therapy vector is a viral expression vector. Viral vectors for use in the present invention typically comprise a viral genome in which a portion of the native sequence has been deleted in order to introduce a heterogeneous polynucleotide without destroying the infectivity of the virus. Due to the specific interaction between virus components and host cell receptors, viral vectors are highly suitable for efficient transfer of genes into target cells. Suitable viral vectors for facilitating gene transfer into a mammalian cell are well known in the art and can be derived from different types of viruses, for example, from a retrovirus, adenovirus, AAV, orthomyxovirus, paramyxovirus, papovavirus, picornavirus, lentivirus, herpes simplex virus, vaccinia virus, pox virus or alphavirus. For an overview of the different viral vector systems, see Nienhuis et al., Hematology, Vol. 16: Viruses and Bone Marrow, N. S. Young (ed.), 353-414 (1993).

For example, retroviral vectors may be used. Retroviral vectors normally function by transducing and integrating the selected polynucleotide into the genome of the target cell. The retroviral vectors can be derived from any of the subfamilies. For example, vectors from Murine Sarcoma Virus, Bovine Leukemia, Virus Rous Sarcoma Virus, Murine Leukemia Virus, Mink-Cell FocusInducing Virus, Reticuloendotheliosis Virus, or Avian Leukosis Virus can be used. The skilled person will be able to combine portions derived from different retroviruses, such as LTRs, tRNA binding sites, and packaging signals to provide a recombinant retroviral vector. These retroviral vectors are then normally used for producing transduction competent retroviral vector particles. Retrovirus vectors can also be constructed for site-specific integration into the DNA of the host cell by incorporating a chimeric integrase enzyme into the retroviral particle.

For gene therapy applications, modification of vectors can include the insertion of constitutive or liver targeted promoters and/or posttranscriptional regulatory elements (e.g., WPRE).

In certain embodiments, the nucleic acid constructs of the present invention may comprise one or more of signal peptide sequences, promoters, enhancers, initiators, terminators and other elements, such as for example polyadenylation (polyA) signals and/or a WPRE sequence. The nucleic acid constructs of the present invention may also comprise nucleotide sequences which facilitate their genetic manipulation, such as restriction sites.

In specific embodiments, the promoter may be selected from, for example, constitutive promoters, and cell/tissue/organ-specific promoters, e.g., Tet-inducible promoters and VP16-LexA promoters. For example, the constitutive promoter can be the cytomegalovirus (CMV) immediate early promoter, CMV early enhancer/chicken β actin (CAG) promoter, or the small nuclear RNA promoters (U1a and U1b), and the cell/tissue/organ-specific promoter can be a liver-specific promoter such as TBG, hAAT, and ApoE-hAAT. In a specific embodiment, the promoter is a CBh promoter, and the terminator is bovine growth hormone polyadenylation signal (BGH pA).

In certain embodiments, the nucleic acid constructs/vectors of the subject invention comprise a control sequence (such as a promoter) operably linked to a nucleotide sequence encoding a fusion of interest, thus, allowing for expression of the fusion of interest in vivo. As used herein, the term “operably linked” refers to a juxtaposition of two or more nucleotide sequences that allows each of said two or more sequences to perform their normal function. Typically, the term operably linked is used to refer to the juxtaposition of a regulatory element (e.g., a promoter, enhancer, polyA signal sequence, WPRE sequence, etc.) and a nucleotide sequence encoding a fusion of interest. For example, an operable linkage between a promoter and a fusion-encoding nucleotide sequence permits the promoter to function to drive the expression of the fusion of interest in vivo.

In certain embodiments, the regulatory element that may be used in the nucleic acid constructs of the present invention is a woodchuck hepatitis virus (WHP) posttranscriptional regulatory element (WPRE). A WPRE is a DNA sequence that, when transcribed into mRNA, creates tertiary structure in the mRNA transcript thereby enhancing the stability of the mRNA and expression of the fusion of interest encoded by the nucleic acid construct/vector. In the nucleic acid constructs of the present invention, the WPRE may be 3′ to the nucleotide sequence encoding the fusion of interest.

In certain embodiments, the regulatory element that may be used in the nucleic acid constructs of the present invention is a polyadenylation (poly(A)) signal sequence. In eukaryotic cells, polyadenylation signal sequences within mRNA transcripts are recognized and processed to add a poly(A) tail consisting of multiple adenosine monophosphates at the 3′ end of the mRNA transcript. The poly(A) tail functions to promote export of the mRNA from the nucleus to the cytoplasm and prevents the degradation of the mRNA, thereby enhancing expressing of the fusion of interest encoded by the nucleic acid construct. In the nucleic acid constructs of the present invention, the polyadenylation signal sequence may be 3′ to the nucleotide sequence encoding the fusion of interest.

In certain embodiments, promoters useful with the subject invention include, for example, the cytomegalovirus immediate early promoter (CMV), the human elongation factor 1-alpha promoter (EFI), the small nuclear RNA promoters (U1a and U1b), α-myosin heavy chain promoter, Simian virus 40 promoter (SV 40), Rous sarcoma virus promoter (RSV), adenovirus major late promoter, β-actin promoter and hybrid regulatory element comprising a CMV enhancer/β-actin promoter.

In certain embodiments, transcriptional enhancer elements that can function to increase levels of transcription from a given promoter can also be included in the vectors of the invention. Enhancers can generally be placed in either orientation, 3′ or 5′, with respect to promoter sequences. In addition to the natural enhancers, synthetic enhancers can be used in the present invention. For example, a synthetic enhancer randomly assembled from SpcS-12-derived elements including muscle-specific elements, serum response factor binding element (SRE), myocyte-specific enhancer factor-1 (MEF-1), myocyte-specific enhancer factor-2 (MEF-2), transcription enhancer factor-1 (TEF-1) and SP-1 may be used in vectors of the invention.

In certain embodiments, to visualize the exogenous gene expression, other optional elements can be introduced in the fusion construct or gene therapy vector such as tag sequences (myc, FLAG, HA, His, and the like), or fluorochromes such as GFP, YFP, and RFP.

In some embodiments, the AAV gene therapy vector comprises a promoter such as CMV and CBh; and a polynucleotide sequence encoding a fusion protein comprising a lectin and a therapeutic agent.

In some embodiments, the AAV gene therapy vector comprises a liver-specific promoter; and a polynucleotide sequence encoding a fusion protein comprising a lectin and a therapeutic agent.

In specific embodiments, the AAV gene therapy vector comprises i) a liver-specific promoter; ii) a polynucleotide sequence encoding a fusion protein comprising a lectin and a therapeutic agent; iii) a terminator such as BGH pA; and iv) optionally, ITR sequences at the 3′ and 5′ ends.

In specific embodiments, the AAV gene therapy vector comprises i) a N-terminal signal peptide sequence; ii) a constitutive promoter such as CMV and CBh; iii) a polynucleotide sequence encoding a fusion protein comprising a lectin and a therapeutic agent; iv) a terminator such as BGH pA; and v) optionally, ITR sequences at the 3′ and 5′ ends.

In certain embodiments, the subject invention provides an AAV vector gene therapy for treating a musculoskeletal disease, wherein the AAV vector comprises a nucleic acid sequence encoding a lectin and a nucleic acid sequence encoding a therapeutic protein. In specific embodiments, the lectin is selected from: 1) B subunits from AB toxins such as ricins, abrins, nigrins, and mistletoe toxins, viscumin toxins, ebulins, pharatoxin, hurin, phasin, and pulchellin; and 2) wheat germ agglutinin, peanut agglutinin, and tomato lectin. In a specific embodiment, the lectin is RTB or NNB.

In specific embodiments, the therapeutic protein is selected from enzymes, proteases, chaperones, and growth factors. In a preferred embodiment, the therapeutic protein is selected from CAPN3, GYS1, POGLUT1, GAA, PYGM, AGL, GBE1, PYGL, PFKM, ALDOA, GYS2, ENO3, DSE, CHST14, PGAM2, GTG1, FKRP, POMT1, CAPN3, NEU1, SMPD1, IDS, NAGLU, SGSH, IDUA, GNS, GALNS, ARSB, HYAL1, MAN2B1, FUCA1, NAGA, CTSA, P3H1, FKBP10, BMP1, WNT1, SPARC, MESD, CRTAP, SERPINH1, SERPINF1 and TENT5A

In specific embodiments, the AAV vector comprises a nucleic acid sequence encoding a fusion protein selected from IDUA:RTB, RTB:GALNS, GAA:RTB, CRTAP:RTB, P3H1:RTB, beta-Gal:RTB, and ARSB:RTB.

In certain embodiments, the AAV vector may further comprise a signal peptide, a promoter, a terminator, and/or one or two Inverted Terminal Repeat (ITR) sequences.

In specific embodiments, the AAV vector is selected from AAV serotypes 1, 2, 3B, 4, 5, 6, 8, and 9. In preferred embodiments, the AAV vector is AAV serotype 8 or 9.

As used herein, an “AAV vector” refers to a recombinant AAV vector that is derived from the wild type AAV by using molecular methods. An AAV vector is distinguished from a wild type (wt) AAV vector because at least a part of the viral genome has been replaced with a transgene, which is a non-native nucleic acid with respect to the wild-type AAV nucleic acid sequence.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralizing antibodies specific for any other AAV serotype. AAV serotypes include AAV1, AAV2, AAV3, AAV4, AAV5, AAV6, AAV7, AAV8, AAV9, AAV10 (AAVrH10) and AAV11, also recombinant serotypes, such as Rec2 and Rec3 identified from primate brain. In the vectors of the invention, the genome may be derived from any AAV serotype. The capsid may also be derived from any AAV serotype. The genome and the capsid may be derived from the same serotype or different serotypes.

Reviews of AAV serotypes may be found in Choi et al (Curr Gene Ther. 2005; 5(3); 299-310) and Wu et al (Molecular Therapy. 2006; 14(3), 316-327). The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065,5AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

Preferably, a gene product of interest is flanked by AAV ITRs on either side. Any AAV ITR may be used in the constructs of the invention, including ITRs from, for example, AAV1, AAV2, AAV4, AAV5, AAV6, AAV8, and/or AAV9.

The term “transgene” is used to refer to a non-native nucleic acid with respect to the AAV nucleic acid sequence. It is used to refer to a polynucleotide that can be introduced into a cell or organism. Transgenes include any polynucleotide, such as a gene that encodes a polypeptide or protein, or a polynucleotide that is not transcribed (e.g., lacks an expression control element, such as a promoter that drives transcription). A transgene is preferably inserted between ITR sequences.

As used herein, “gene therapy” is the insertion of nucleic acid sequences (e.g., a transgene) into an individual's cells and/or tissues to treat a disease. For example, the transgene can be a functional mutant allele that replaces or supplements a defective one. Gene therapies also include insertion of transgene that are inhibitory in nature, i.e., that inhibit, decrease or reduce expression, activity or function of an endogenous gene or protein, such as an undesirable or aberrant (e.g., pathogenic) gene or protein. Such transgenes may be exogenous. An exogenous molecule or sequence is understood to be molecule or sequence not normally occurring in the cell, tissue and/or individual to be treated.

The compounds of the subject invention, and compositions comprising them, can also be administered utilizing liposome and nanotechnology, slow-release capsules, implantable pumps, and biodegradable containers, and orally or intestinally administered intact plant cells expressing the therapeutic product. These delivery methods can, advantageously, provide a uniform dosage over an extended period.

Compounds of the subject invention can be formulated according to known methods for preparing physiologically acceptable compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the compound is combined with a suitable carrier in order to facilitate effective administration of the composition.

The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional physiologically acceptable carriers and diluents which are known to those skilled in the art. Examples of carriers or diluents for use with the subject compounds include ethanol, dimethyl sulfoxide, glycerol, alumina, starch, saline, and equivalent carriers and diluents. To provide for the administration of such dosages for the desired therapeutic treatment, compositions of the invention will advantageously comprise between about 0.1% and 99%, and especially, 1 and 15% by weight of the total of one or more of the subject compounds based on the weight of the total composition including carrier or diluent.

Compounds and agents of the invention, and compositions thereof, may be locally administered at one or more anatomical sites, optionally in combination with a pharmaceutically acceptable carrier such as an inert diluent. Compounds and agents of the invention, and compositions thereof, may be systemically administered, such as intravenously or orally, optionally in combination with a pharmaceutically acceptable carrier such as an inert diluent, or an assimilable edible carrier for oral delivery. They may be enclosed in hard- or soft-shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Compounds and agents, and compositions of the invention, including pharmaceutically acceptable salts or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent of the invention in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds and agents and pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The present invention also provides pharmaceutical compositions comprising a compound and/or agent of the invention in combination with a pharmaceutically acceptable carrier. Pharmaceutical compositions adapted for oral, topical or parenteral administration, comprising an amount of a compound constitute a preferred embodiment of the invention. The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

To provide for the administration of such dosages for the desired therapeutic treatment, in some embodiments, pharmaceutical compositions of the invention can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

The subject invention also concerns kits comprising a composition comprising a compound and/or agent and/or polynucleotide of the invention in one or more containers. Kits of the invention can optionally include pharmaceutically acceptable carriers and/or diluents.

In one embodiment, a kit of the invention includes one or more other components, adjuncts, or adjuvants as described herein. In one embodiment, a kit of the invention includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent and/or polynucleotide of the invention is provided in the kit as a solid, such as a tablet, pill, or powder form. In another embodiment, a compound and/or agent and/or polynucleotide of the invention is provided in the kit as a liquid or solution. In one embodiment, the kit comprises an ampoule or syringe containing a compound and/or agent of the invention in liquid or solution form.

Mammalian species that benefit from the disclosed methods include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Other species that may benefit from the disclosed methods include fish, amphibians, avians, and reptiles. As used herein, the terms “patient” and “subject” are used interchangeably and are intended to include such human and non-human species. Likewise, in vitro methods of the present invention can be carried out on cultured cells or tissues of such human and non-human species.

The subject invention also provides bacterial cells, animal tissue, animal cells (e.g., human cells), plants, plant tissue, and plant cells that comprise or express a polynucleotide or the protein encoded by the polynucleotide of the invention, or a fragment or variant thereof. Plant tissue includes, but is not limited to, seed, scion, and rootstock. Plants within the scope of the present invention include monocotyledonous plants, such as, for example, rice, wheat, barley, oats, rye, sorghum, maize, sugarcane, pineapple, onion, bananas, coconut, lilies, turfgrasses, and millet. Plants within the scope of the present invention also include dicotyledonous plants, such as, for example, tomato, cucumber, squash, peas, alfalfa, melon, chickpea, chicory, clover, kale, lentil, soybean, beans, tobacco, potato, sweet potato, yams, cassava, radish, broccoli, spinach, cabbage, rape, apple trees, citrus (including oranges, mandarins, grapefruit, lemons, limes and the like), grape, cotton, sunflower, strawberry, lettuce, and hop. Herb plants containing a polynucleotide of the invention are also contemplated within the scope of the invention. Herb plants include parsley, sage, rosemary, thyme, and the like. In one embodiment, a plant, plant tissue, or plant cell is a transgenic plant, plant tissue, or plant cell. In another embodiment, a plant, plant tissue, or plant cell is one that has been obtained through a breeding program.

Polynucleotides encoding a fusion product of the present invention can be provided in an expression construct. Expression constructs of the invention generally include regulatory elements that are functional in the intended host cell in which the expression construct is to be expressed. Thus, a person of ordinary skill in the art can select regulatory elements for use in bacterial host cells, yeast host cells, plant host cells, insect host cells, mammalian host cells, and human host cells. Regulatory elements include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

An expression construct of the invention can comprise a promoter sequence operably linked to a polynucleotide sequence of the invention, for example a sequence encoding a fusion polypeptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

If the expression construct is to be provided in or introduced into a plant cell, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used.

Other promoters that can be used for expression constructs in plants include, for example, prolifera promoter, Ap3 promoter, heat shock promoters, T-DNA1′- or 2′-promoter of A. tumefaciens, polygalacturonase promoter, chalcone synthase A (CHS-A) promoter from petunia, tobacco PR-1a promoter, ubiquitin promoter, actin promoter, alcA gene promoter, pin2 promoter (Xu et al., 1993), maize WipI promoter, maize trpA gene promoter (U.S. Pat. No. 5,625,136), maize CDPK gene promoter, and RUBISCO SSU promoter (U.S. Pat. No. 5,034,322) can also be used.

Tissue-specific promoters, for example, fruit-specific promoters, such as the E8 promoter of tomato (accession number: AF515784; Good et al. (1994)) can be used. Fruit-specific promoters such as flower organ-specific promoters can be used with an expression construct of the present invention for expressing a polynucleotide of the invention in the flower organ of a plant. Examples of flower organ-specific promoters include any of the promoter sequences described in U.S. Pat. Nos. 6,462,185; 5,639,948; and 5,589,610. Seed-specific promoters such as the promoter from a β-phascolin gene (for example, of kidney bean) or a glycinin gene (for example, of soybean), and others, can also be used. Endosperm-specific promoters include, but are not limited to, MEG1 (EPO application No. EP1528104) and those described by Wu et al. (1998), Furtado et al. (2002), and Hwang et al. (2002). Root-specific promoters, such as any of the promoter sequences described in U.S. Pat. No. 6,455,760 or U.S. Pat. No. 6,696,623, or in published U.S. patents application Nos. 20040078841; 20040067506; 20040019934; 20030177536; 20030084486; or 20040123349, can be used with an expression construct of the invention. Constitutive promoters (such as the CaMV, ubiquitin, actin, or NOS promoter), developmentally-regulated promoters, and inducible promoters (such as those promoters than can be induced by heat, light, hormones, or chemicals) are also contemplated for use with polynucleotide expression constructs of the invention.

Expression constructs of the invention may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment.

Targeting gene products to an intended cellular and/or extracellular destination through the use of an operably linked signal peptide sequence is contemplated for use with the polypeptides of the invention. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

DNA sequences that direct polyadenylation of mRNA transcribed from the expression construct can also be included in the expression construct, and include, but are not limited to, an octopine synthase or nopaline synthase signal. The expression constructs of the invention can also include a polynucleotide sequence that directs transposition of other genes, i.e., a transposon.

Polynucleotides of the present invention can be composed of either RNA or DNA. Preferably, the polynucleotides are composed of DNA. The subject invention also encompasses those polynucleotides that are complementary in sequence to the polynucleotides disclosed herein. Polynucleotides and polypeptides of the invention can be provided in purified or isolated form.

Because of the degeneracy of the genetic code, a variety of different polynucleotide sequences can encode polypeptides and enzymes of the present invention. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, polypeptides and enzymes of the subject invention. These variant or alternative polynucleotide sequences are within the scope of the subject invention. As used herein, references to “essentially the same” sequence refers to sequences which encode amino acid substitutions, deletions, additions, or insertions which do not materially alter the functional activity of the polypeptide encoded by the polynucleotides of the present invention. Allelic variants of the nucleotide sequences encoding a wild type polypeptide of the invention are also encompassed within the scope of the invention.

Substitution of amino acids other than those specifically exemplified or naturally present in a wild type polypeptide or enzyme of the invention are also contemplated within the scope of the present invention. For example, non-natural amino acids can be substituted for the amino acids of a polypeptide, so long as the polypeptide having the substituted amino acids retains substantially the same biological or functional activity as the polypeptide in which amino acids have not been substituted.

Examples of non-natural amino acids include, but are not limited to, ornithine, citrulline, hydroxyproline, homoserine, phenylglycine, taurine, iodotyrosine, 2,4-diaminobutyric acid, α-amino isobutyric acid, 4-aminobutyric acid, 2-amino butyric acid, γ-amino butyric acid, ε-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form. Allelic variants of a protein sequence of a wild type polypeptide or enzyme of the present invention are also encompassed within the scope of the invention.

Amino acids can be generally categorized in the following classes: non-polar, uncharged polar, basic, and acidic. Conservative substitutions whereby a polypeptide or enzyme of the present invention having an amino acid of one class is replaced with another amino acid of the same class fall within the scope of the subject invention so long as the polypeptide having the substitution still retains substantially the same biological or functional activity (e.g., enzymatic) as the polypeptide that does not have the substitution. Polynucleotides encoding a polypeptide or enzyme having one or more amino acid substitutions in the sequence are contemplated within the scope of the present invention. Table 1 below provides a listing of examples of amino acids belonging to each class.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

The subject invention also concerns variants of the polynucleotides of the present invention that encode functional polypeptides of the invention. Variant sequences include those sequences wherein one or more nucleotides of the sequence have been substituted, deleted, and/or inserted. The nucleotides that can be substituted for natural nucleotides of DNA have a base moiety that can include, but is not limited to, inosine, 5-fluorouracil, 5-bromouracil, hypoxanthine, 1-methylguanine, 5-methylcytosine, and tritylated bases. The sugar moiety of the nucleotide in a sequence can also be modified and includes, but is not limited to, arabinose, xylulose, and hexose. In addition, the adenine, cytosine, guanine, thymine, and uracil bases of the nucleotides can be modified with acetyl, methyl, and/or thio groups. Sequences containing nucleotide substitutions, deletions, and/or insertions can be prepared and tested using standard techniques known in the art.

Fragments and variants of a polypeptide or enzyme of the present invention can be generated as described herein and tested for the presence of biological or enzymatic function using standard techniques known in the art. Thus, an ordinarily skilled artisan can readily prepare and test fragments and variants of a polypeptide or enzyme of the invention and determine whether the fragment or variant retains functional or biological activity (e.g., enzymatic activity) relative to full-length or a non-variant polypeptide.

Polynucleotides and polypeptides contemplated within the scope of the subject invention can also be defined in terms of more particular identity and/or similarity ranges with those sequences of the invention specifically exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. Allelic variations of the exemplified sequences also fall within the scope of the subject invention. The polynucleotide sequence includes both the sense and antisense strands either as individual strands or in the duplex.

Techniques for transforming plant cells with a polynucleotide or gene are known in the art and include, for example, Agrobacterium infection, transient uptake and gene expression in plant seedlings, biolistic methods, electroporation, calcium chloride treatment, PEG-mediated transformation, etc. U.S. Pat. No. 5,661,017 teaches methods and materials for transforming an algal cell with a heterologous polynucleotide. Transformed cells can be selected, redifferentiated, and grown into plants that contain and express a polynucleotide of the invention using standard methods known in the art. The seeds and other plant tissue and progeny of any transformed or transgenic plant cells or plants of the invention are also included within the scope of the present invention.

The subject invention also provides cells transformed with a polynucleotide of the present invention encoding a polypeptide or enzyme of the invention. In one embodiment, the polynucleotide sequence of the invention is provided in an expression construct of the invention. The transformed cell can be a prokaryotic cell, for example, a bacterial cell such as E. coli or B. subtilis, or the transformed cell can be a eukaryotic cell, for example, a plant cell, including protoplasts, or an animal cell. Plant cells include, but are not limited to, dicotyledonous, monocotyledonous, and conifer cells. Animal cells include human cells, mammalian cells, avian cells, and insect cells. Mammalian cells include, but are not limited to, COS, 3T3, and CHO cells.

Single letter amino acid abbreviations are defined in Table 2.

TABLE 2

| Letter Symbol | Amino Acid |
|---|---|
| A | Alanine |
| B | Asparagine or |
| | aspartic acid |
| C | Cysteine |
| D | Aspartic Acid |
| E | Glutamic Acid |
| F | Phenylalanine |
| G | Glycine |
| H | Histidine |
| I | Isoleucine |
| K | Lysine |
| L | Leucine |
| M | Methionine |
| N | Asparagine |
| P | Proline |
| Q | Glutamine |
| R | Arginine |
| S | Serine |
| T | Threonine |
| V | Valine |
| W | Tryptophan |
| Y | Tyrosine |
| Z | Glutamine or |
| | glutamic acid |

In one embodiment, the subject invention provides a method for treating a musculoskeletal disease in a subject, the method comprising administering the fusion protein/fusion entity of the subject invention or the composition of the subject invention to the subject in need thereof.

In one embodiment, the subject invention provides a method for treating a musculoskeletal condition, the method comprising administering, to a subject in need thereof, a) a fusion construct comprising a lectin and a therapeutic agent, or b) a polynucleotide sequence encoding a fusion construct comprising a lectin and a therapeutic agent.

In one embodiment, the subject invention provides a method for treating a musculoskeletal condition, the method comprising administering, to a subject in need thereof, a composition comprising a) a fusion construct comprising a lectin and a therapeutic agent, or b) a polynucleotide sequence encoding a fusion construct comprising a lectin and a therapeutic agent.

In some embodiments, the musculoskeletal disease is selected from genetic diseases with skeletal pathology, lysosomal diseases with skeletal pathology, collagen-associated diseases, bone diseases, and genetic muscle diseases. In specific embodiment, the musculoskeletal disease is selected from Muscular Dystrophy, Limb-Girdle, Autosomal Recessive 1; Glycogen Storage Disease; Muscular Dystrophy, Limb-Girdle, Autosomal Recessive 21; Ehlers-Danlos Syndrome; Muscular Dystrophy-Dystroglycanopathy; Autosomal Recessive Limb-Girdle Muscular Dystrophy Type 2a; Neuraminidase Deficiency; Niemann-Pick Disease, Type B; Mucopolysaccharidosis; Hurler Syndrome; Mannosidosis, Alpha B, Lysosomal; Fucosidosis; Schindler Disease, Type I; Osteogenesis Imperfecta.

In certain embodiments, the diseases, preferably, musculoskeletal diseases, are characterized by deficiency in one or more proteins selected from, for example, CAPN3, GYS1, POGLUT1, GAA, PYGM, AGL, GBE1, PYGL, PFKM, ALDOA, GYS2, ENO3, DSE, CHST14, PGAM2, GTG1, FKRP, POMT1, CAPN3, NEU1, SMPD1, IDS, NAGLU, SGSH, IDUA, GNS, GALNS, ARSB, HYAL1, MAN2B1, FUCA1, NAGA, P3H1, FKBP10, BMP1, WNT1, SPARC, MESD, CRTAP, SERPINH1, SERPINF1 and TENT5A.

In specific embodiments, the diseases, preferably, musculoskeletal diseases, are characterized by deficiency in one or more proteins selected from, for example, IDUA, GALNS, GAA, CRTAP, P3H1, beta-Gal, and ARSB.

In certain embodiments, the method of the subject invention further comprises evaluating the level of one or more proteins selected from, for example, CAPN3, GYS1, POGLUT1, GAA, PYGM, AGL, GBE1, PYGL, PFKM, ALDOA, GYS2, ENO3, DSE, CHST14, PGAM2, GTG1, FKRP, POMT1, CAPN3, NEU1, SMPD1, IDS, NAGLU, SGSH, IDUA, GNS, GALNS, ARSB, HYAL1, MAN2B1, FUCA1, NAGA, P3H1, FKBP10, BMP1, WNT1, SPARC, MESD, CRTAP, SERPINH1, SERPINF1 and TENT5A before and/or after the administering step.

In certain embodiments, the method of the subject invention further comprises examining musculoskeletal cells (e.g., chondrocytes, and muscle myocytes) for the presence of the fusion construct of the subject invention or for the level of one or more proteins selected from, for example, CAPN3, GYS1, POGLUT1, GAA, PYGM, AGL, GBE1, PYGL, PFKM, ALDOA, GYS2, ENO3, DSE, CHST14, PGAM2, GTG1, FKRP, POMT1, CAPN3, NEU1, SMPD1, IDS, NAGLU, SGSH, IDUA, GNS, GALNS, ARSB, HYAL1, MAN2B1, FUCA1, NAGA, P3H1, FKBP10, BMP1, WNT1, SPARC, MESD, CRTAP, SERPINH1, SERPINF1 and TENT5A. In specific embodiments, the examining step may comprise accessing enzyme activity of the fusion construct in musculoskeletal cells, and/or detecting the location of the fusion construct. In specific embodiments, the examining step may further comprise labeling the fusion construct and/or detecting a signal from the label.

In certain embodiments, the method of the subject invention further comprises detecting enzymes activity levels in, for example, liver, spleen, kidney, heart, lung, eye, trachea, bone, and bone marrow.

In certain embodiments, the method of the subject invention further comprises detecting levels of bone remodeling biomarkers such as Osteocalcin, Collagen 10a1, Bone Morphogenetic Protein 2, Acid Phosphatase 1, and Integring Binding Asialoprotein.

In one embodiment, the subject invention provides a method for improving bone remodeling, the method comprising administering, to a subject in need thereof, a fusion protein/entity comprising a lectin and the therapeutic agent or a polynucleotide sequence encoding a fusion construct comprising a lectin and a therapeutic agent.

In one embodiment, the subject invention provides a method for improving bone remodeling, the method comprising administering, to a subject in need thereof, a composition comprising a fusion protein/entity comprising a lectin and the therapeutic agent or a polynucleotide sequence encoding a fusion construct comprising a lectin and a therapeutic agent.

In one embodiment, the subject invention provides a method for treating MPS I, the method comprising administering, to a subject in need thereof, a) a fusion construct comprising a lectin and a therapeutic agent, or b) a polynucleotide sequence encoding a fusion construct comprising a lectin and a therapeutic agent.

In one embodiment, the subject invention provides a method for treating MPS I, the method comprising administering, to a subject in need thereof, a composition comprising a) a fusion construct comprising a lectin and a therapeutic agent, or b) a polynucleotide sequence encoding a fusion construct comprising a lectin and a therapeutic agent.

In one embodiment, the subject invention also provides a method for delivering a therapeutic agent to cells of the musculoskeletal system of a subject, the method comprising administering, to the subject in need, a fusion construct comprising a lectin (e.g., plant lectin) and the therapeutic agent, or a polynucleotide sequence encoding a fusion construct comprising a lectin and a therapeutic agent.

In one embodiment, the subject invention provides a method for delivering a therapeutic agent to a cell of the musculoskeletal system of a subject, in the presence of immune components against the therapeutic agent, the method comprising administering a fusion construct comprising a lectin (e.g., plant lectin) and the therapeutic agent or a polynucleotide sequence encoding a fusion construct comprising a lectin and a therapeutic agent to the subject in need thereof, wherein the immune components against the therapeutic agent do not compromise the therapeutic response in targeted tissues. In specific embodiments, the immune components are antibodies or antisera that bind to one or more epitopes of said agent.

One aspect of the subject invention provides a “delivery-enhanced” ERT drug for Morquio A that incorporates an RTB delivery module (RTB:GALNS) and directly targets the key limitations of current Morquio ERT treatments.

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof), such as “comprising,” “comprises,” and “comprise,” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

Following are examples that illustrate procedures for practicing the invention. These examples should not be construed as limiting. All percentages are by weight and all solvent mixture proportions are by volume unless otherwise noted.

### EXAMPLES

### Example 1-Lectin-Based Carriers Provide New Mechanisms of ERT Uptake and Lysosomal Trafficking

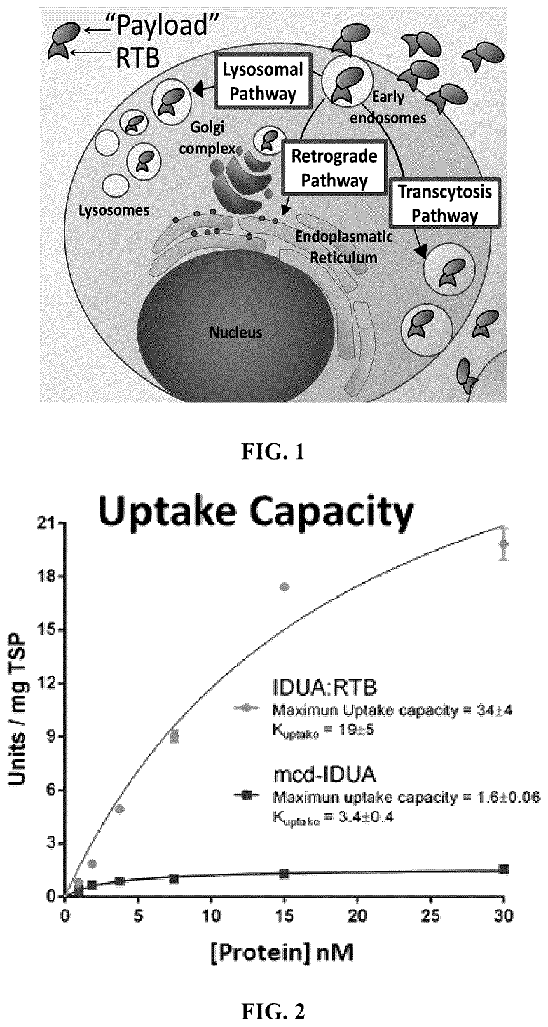

The subject invention encompasses the use of the RTB plant lectin as a novel carrier for lysosomal enzymes^{15-17,27}. RTB is the non-toxic carbohydrate-binding B subunit of the plant type II AB toxin, ricin. RTB facilitates uptake by targeting cell surface glycoproteins and glycolipids with β-1,4-linked galactose or galactosamine residues. These are abundant on mammalian cells^{18,19}, providing access to virtually all relevant cell types^{20,21}. RTB enters cells by at least 6 different endocytic routes²²⁻²⁵. Upon endocytosis, RTB traverses preferentially to lysosomes (FIG. 1), cycles back to the cell membrane (transcytosis pathway), or exploits the “retrograde” pathway (<5%) to the ER 19.26. Thus, the uptake and transcytosis dynamics of RTB as an enzyme carrier may result in higher drug efficacy (i.e., lower doses) and greater potential to traverse multiple cell layers to gain access to disease-critical cells.

To determine if RTB showed efficacy in ERT delivery, the inventors have produced and tested RTB-lysosomal enzyme fusions for MPS I, MPS IIIA, and GM1 gangliosidosis, lysosomal diseases showing systemic pathologies. Initial breakthroughs, therefore, focused on RTB's ability to delivery enzyme across the blood brain barrier to treat the brain27. RTB targets enzymes to bone and connective tissues—the key unmet need for multiple metabolic disorders. Assessment of skeletal correction was done in the MPS I model using the RTB fusion product α-L-iduronidase:RTB (IDUA:RTB), the most advanced RTB-ERT prototype. Like other multiple metabolic disorders, MPS I present with significant skeletal pathologies. Preclinical studies with IDUA:RTB and the idua-mouse model (most severe ‘Hurler’ phenotype) highlight key advantages of RTB-mediated delivery in the efficiency of cell uptake: correction of visceral, CNS and skeletal disease phenotypes, and the ability to maintain efficacy in the presence of anti-drug antibodies-key features important for developing a broadly effective drugs for other metabolic disorders with musculoskeletal pathologies.

### Example 2-RTB Provides Higher Drug Uptake and Transcytosis in Cells

RTB triggers active and efficient endocytosis into mammalian cells. In contrast to receptor-mediated mechanisms, RTB's absorptive-mediated uptake is not limited by abundance or cycling kinetics associated with specific surface receptors and does not compete with receptor ligands¹⁷. Uptake studies in human fibroblasts demonstrate that, although neither mannan nor M6P in media inhibits RTB uptake, cell uptake is completely inhibited by lactose, demonstrating that uptake depends almost exclusively on the lectin affinity²⁷. In contrast to mcd-IDUA, which is limited by the saturation of receptors limits the amount of med-IDUA enzyme delivered to cells, the capacity for the RTB carrier to mobilize active enzyme into cells is significantly greater (FIG. 2); the maximum uptake capacity was 21-fold higher at saturation point. Similarly, RTB:GALNS does not show saturation in this concentration range (FIG. 11A).

### Example 3-RTB Delivers Enzymes to Hard-to-Treat Tissues and Corrects Lysosomal Pathologies

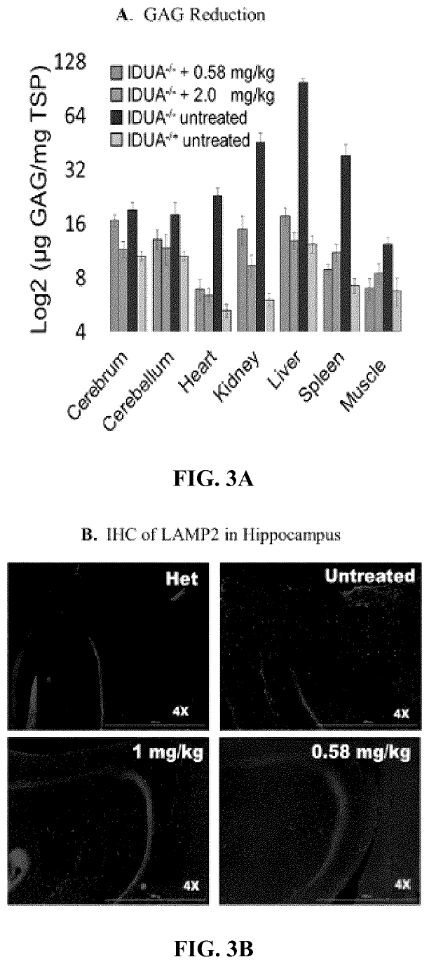

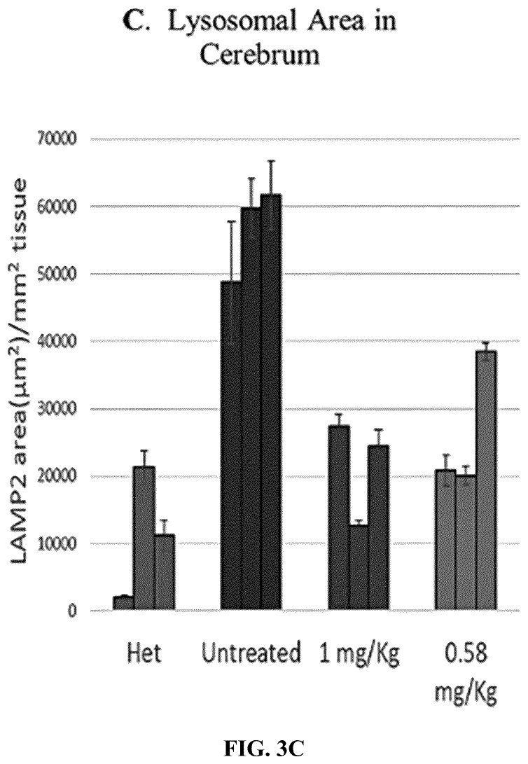

MPS I mice, receiving eight weekly treatments of IDUA:RTB starting at 8 weeks of age, were analyzed for IDUA activity and glycosaminoglycan (GAG; MPS I disease substrate) levels. At the endpoint, GAG levels were corrected to wild-type (WT) levels in all tissues tested (FIG. 3A). Treatment of CNS pathologies was further demonstrated by correction of lysosomal volume (LAMP2) associated with the presence of storage material in the brain (FIGS. 3B and 3C) and normalization of learning/memory deficits assessed by behavioral tests¹⁷. The ability of RTB to deliver fused enzymes across the blood-brain barrier to treat the CNS may be important in treating later stage pathologies of MPS I when impacts on neurocognitive acuity occurs.

### Example 4-RTB Delivers Associated Proteins to Multiple Cell Types of Mesenchymal Lineage

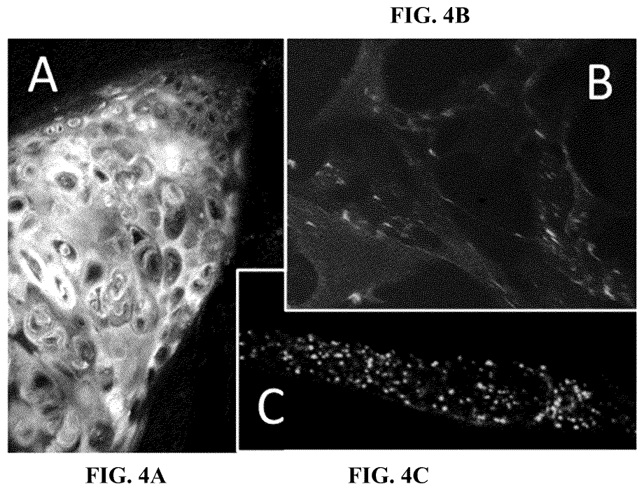

RTB was fused to either green (EGFP) or red (DsRed) fluorescent proteins to demonstrate lectin-mediated uptake into cells important for correction of bone and connective tissues (FIG. 4). RTB:EGFP (FIG. 4B) uptake was seen in osteogenic human mesenchymal stem cells. RTB:EGFP was also used to treat human lung tissues. In addition to uptake into lung macrophages, airway epithelial cells, and lung smooth muscle cells, the most dramatic localization was to chondrocytes of the cartilage plate of bronchial walls (FIG. 4A) with subsequent analyses showing localization surrounding the nucleus indicating uptake as well as strong surface binding.

### Example 5-Bone Pathology is Corrected by RTB:Enzyme Fusions

In MPS I skeletal tissue, elevated GAG levels trigger a cascade of events that leads to aberrant cartilage and bone development. Elevated expression of inflammatory molecules²⁸, impaired cell-to-cell signaling²⁹, enhanced cell proliferation, inhibition of osteoclast proteases³⁰, and apoptosis³¹ cause alterations in the growth plate and structural bone remodeling leading to dysostosis multiplex^{5,31}. Current ERTs fail to prevent the progression of bone disease in patients or animal models^{29,32,33}. The potential of RTB lectin-based carrier to correct bone pathology was tested in the MPS I idua^{-/-} mouse model. These mice display an increased trabecular bone volume and thicker cortical bone compared to WT mice^{32,34}, with initial physical abnormalities beginning at 16 to 24 weeks of age and highly evident skeletal dysplasia³⁴ by 37 weeks. Because morphological differences are only detected in older mice, molecular biomarkers were used to assess therapeutic effects of IDUA:RTB during the bone growth phase (3-8 weeks of age). Elevated GAG levels in the growth plate elicit gene expression of inflammatory cytokines and matrix metalloproteinases (e.g., Mmp3) that contribute to the progression of joint and bone pathology^{31,35}. Elevated Mmp3 expression can be detected as early as 5 weeks of age in MPS I mice³⁵.

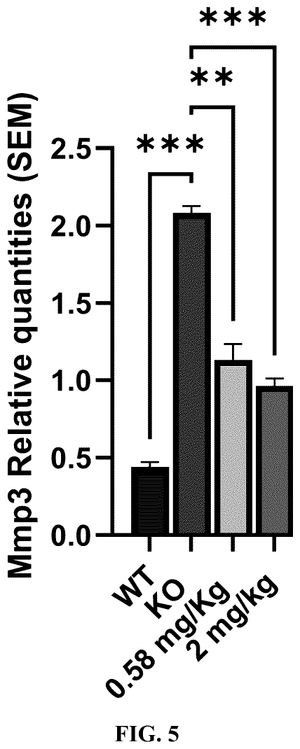

Three-week-old mice (n=5) were treated weekly for five weeks with two different doses of IDUA:RTB (tail vein injection; 0.58 or 2 mg IDUA equivalents/kg) with aged-matched untreated controls carried in parallel. RNA was extracted from the proximal sections of femurs36 and expression levels were quantified by RTq-PCR. Data was normalized (qBase+software) using Canx, Cycl and Actb, genes identified to have more stability in bone samples37. A significant dose-dependent normalization of Mmp3 expression levels was found in mice treated with IDUA:RTB (FIG. 5), suggesting early treatment may aid to prevent the development of bone pathogenesis seen in later disease stages.

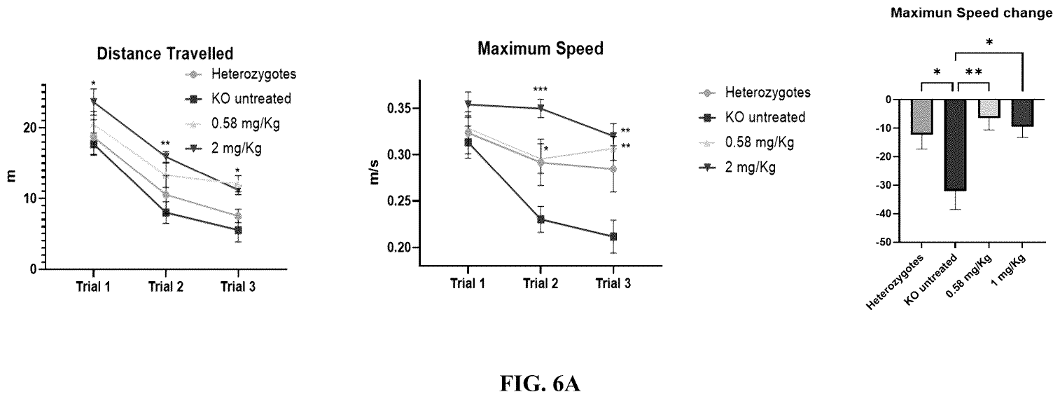

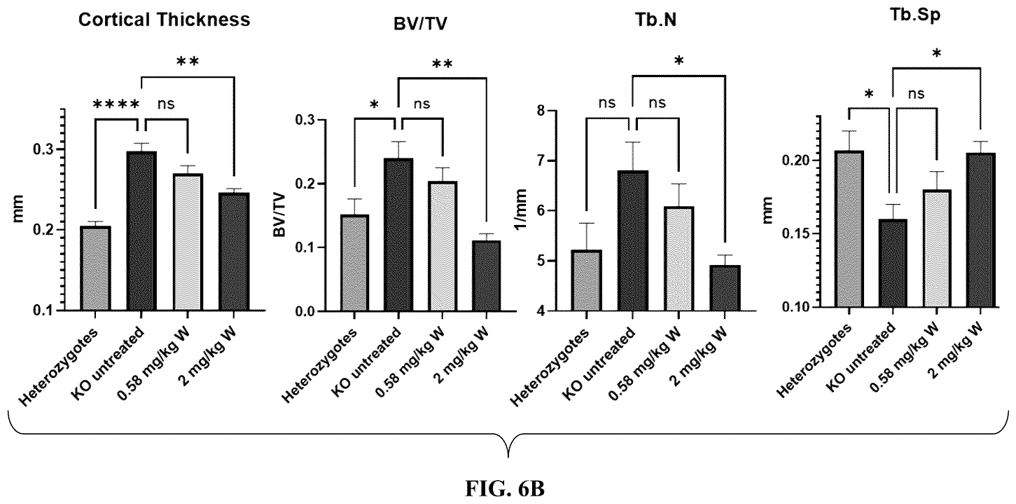

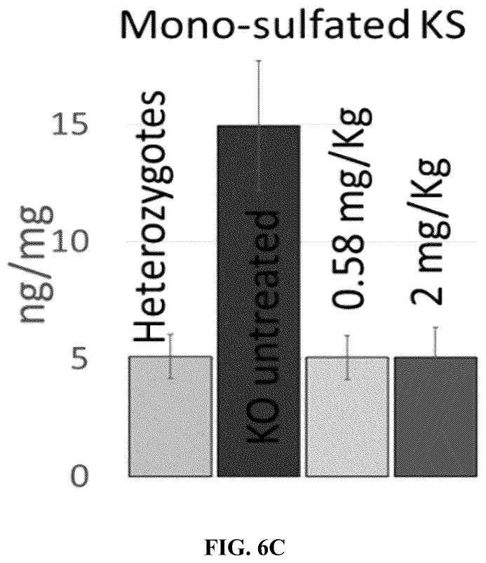

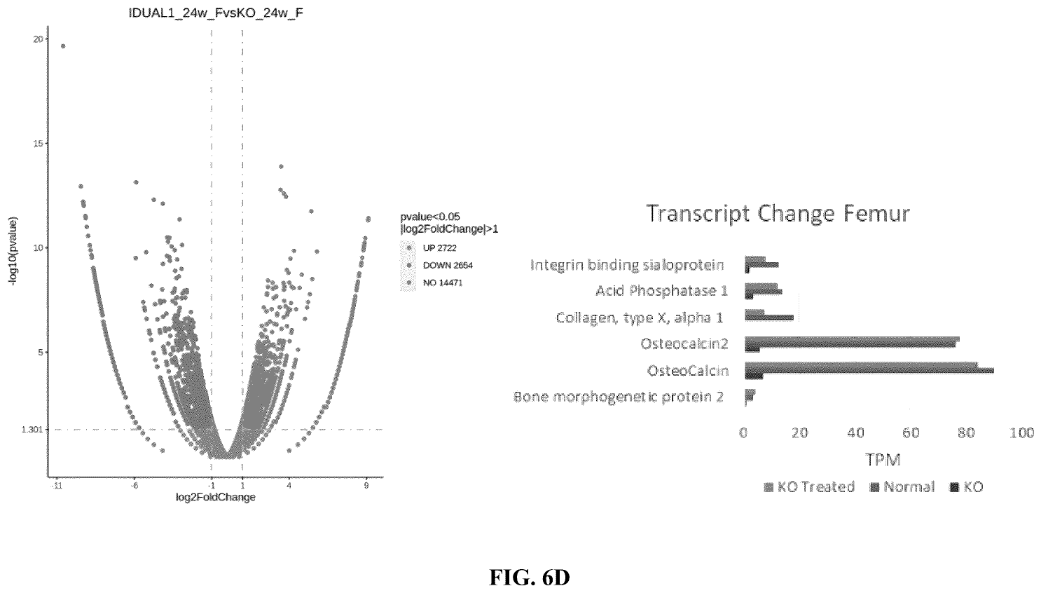

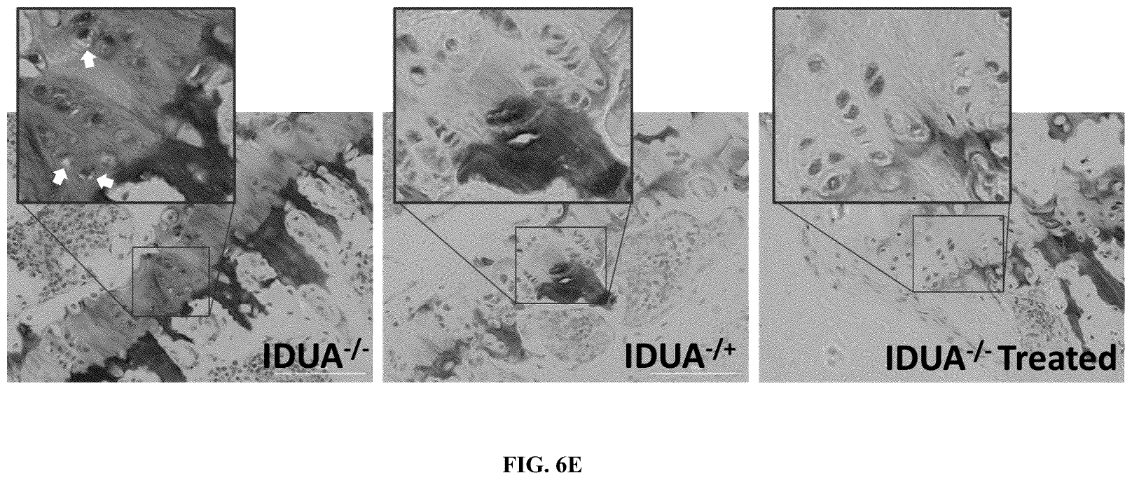

Progression of the MPS I bone disease compromises mobility and endurance in this animal model. Long-term effects of IDUA:RTB administration were tested in MPS I mice with treatment starting at 3 weeks of age. Two doses (0.58 or 2 mg IDUA equivalents/Kg) were administered weekly for 21 weeks to male mice (n=6). Open field test was performed before the last dose using three 5 min test trials for each subject. Trials were video-captured and analyzed with Anymaze® software and maximum speed and total distance travelled in each trial were calculated. All IDUA:RTB-treated mice performed equally or better than untreated normal mice (WT), suggesting normalization of mobility restraints caused by the bone pathology in KO mice (FIG. 6A).

At harvest (24 wk old), tibias were analyzed for micro-CT architecture of trabecular and cortical bone. As described previously, idua^{-/-} mice display thicker cortical bones and higher trabecular bone volume fraction (BV/TV)^{32,34,38}. The trabecular number per length (Tb.N) is increased by the pathology and as a result the trabecular separation (Tb.Sp) is reduced. Significant dose-dependent correction was seen in mice treated with weekly doses of IDUA:RTB (FIG. 6B). IDUA:RTB prevented the thickening of cortical bone and the increase of the trabecular bone volume fraction. The trabecular number and separation stayed at normal levels confirming that the administration of the drug prevented bone disease progression. Mono-sulfated KS level in serum is a biomarker of skeletal dysplasia severity in the MPS I mouse model^{34,38}. The plasma content of KS was measured by tandem mass spectrometry³⁹.

All IDUA:RTB treated mice showed a significant reduction of this skeletal dysplasia biomarker in plasma (FIG. 6C). Serum levels of heparan sulfate were also measured; treated mice showed complete correction, reaching WT levels of both O- and N-sulfated heparan sulfate (data not shown). RNA was isolated from femoral tissue and a pool sample was used for RNA library preparation. The library was sequenced using Illumina PE150 for transcriptome analysis. A volcano plot was generated for transcripts with significant fold-change in KO mice treated with weekly infusions of 0.58 mg/kg of IDUA:RTB compared to untreated KO. A total of 2722 genes were upregulated and 2654 downregulated, evidencing a response on bone transcriptome. Of importance, a significant increase on transcript per million (TPM) of bone remodeling biomarkers such as Osteocalcin, Collagen 10a1, Bone Morphogenetic Protein 2, Acid Phosphatase 1, and Integring Binding Asialoprotein were noted (FIG. 6D).

Untreated MPS I KO mice at 24 weeks of age exhibited GAG storage vacuoles in the chondrocytes of the growth plate of the femur and tibia (Russell, C et al. 1998), as well as increased hyaline cartilage accumulation. Tibias from treated mice were processed for histology and stained with toluidine blue. A significant reduction on cartilage build up was noted in treated mice compared to untreated controls, almost reaching normal cartilage content seen in normal mice. Chondrocytes embedded in the cartilage of the growth plate did not display aberrant GAG buildup within the lysosomes, demonstrating efficient delivery of the IDUA enzyme across cartilage and correcting the cells in this critical site of pathology (FIG. 6E).

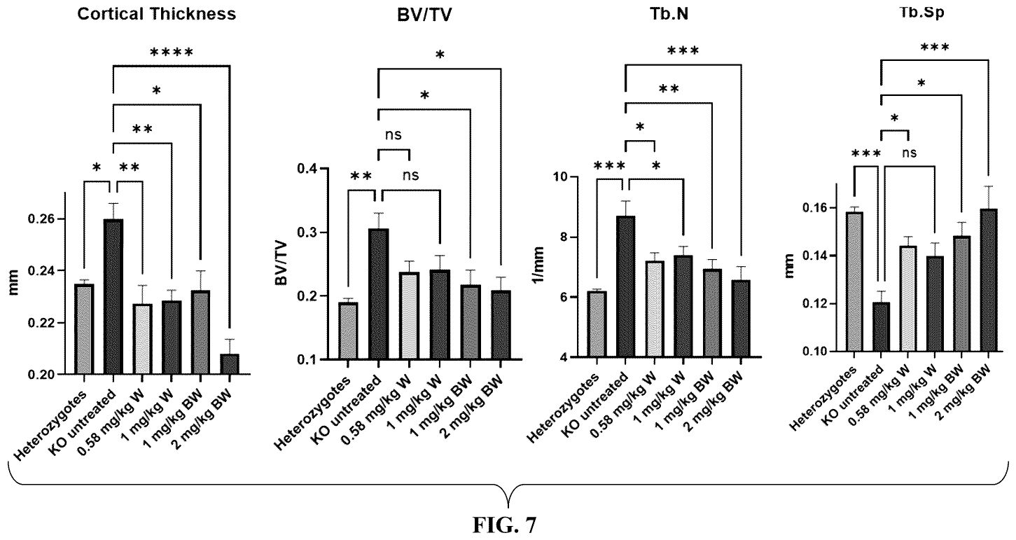

In a separate experiment, the efficacy of IDUA:RTB treatment during the bone remodeling phase was tested using adult MPS I mice (FIG. 7). Treatment was initiated in 8 weeks old mice and four dose/frequency combinations were tested. Weekly doses of 0.58 or 1 mg/kg were compared to biweekly (every other week) administrations of 1 or 2 mg/kg. Mice were harvested at 17 weeks of age and micro-CT architecture was analyzed in tibias as described above. The “normalization” to WT architecture was achieved even after the complete development of bone growth. Results herein demonstrate that IDUA:RTB aid normalization during bone remodeling and has a therapeutic effect over the pathological metabolic cascade created during the early stages of the disease.

### Example 6-Muscle Pathology is Corrected by RTB:Enzyme Fusions

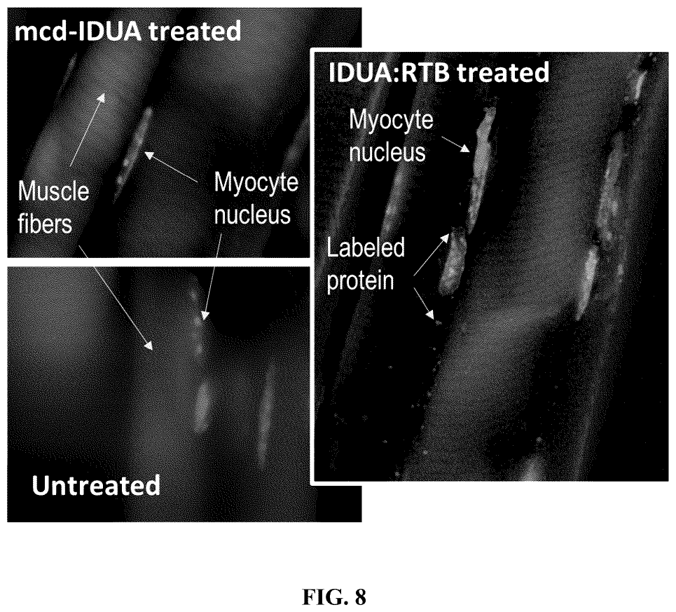

ERT delivery to muscle myocytes remains a major challenge. RTB-mediated delivery overcome this. In the MPS I model, muscle GAG levels were reduced in biceps femoris muscles of IDUA:RTB-treated mice (FIG. 8). Delivery of product to muscle cells was confirmed by histology techniques using fluorescently labeled IDUA:RTB or mammalian cell derived med-IDUA (Aldurazyme equivalent) injected into MPS I mice.

Infrared-fluorescence-labeled IDUA:RTB or medIDUA (Aldurazyme equivalent) was administrated to MPS I mice by tail-vein injection (2 mg/kg). Bicep femoris muscle samples were collected and processed for paraffin embedding. Samples were stained with DAPI (nuclei) and imaged. As shown in FIG. 8, IDUA:RTB, but not med-IDUA, was detected as punctate structures typical of lysosomes within myocytes consistent with lysosomal delivery.

### Example 7-RTB is Non-Immunogenic and Mitigates Anti-Drug Immunogenicity

In the mouse trials described above, the development of ADA to IDUA:RTB was analyzed. Antibodies directed against IDUA epitopes were observed. Subsequent analyses indicated that anti-IDUA antibodies were IgG1s and were not directed toward IDUA:RTB glycans¹⁷. In contrast, no anti-RTB IgGs were detected in the same serum samples. These results show that RTB is advantageously non-immunogenic.

Uptake of IDUA:RTB is not hindered by the presence of anti-IDUA neutralizing antibodies. Neutralizing serum from Aldurazyme®-immunized dogs inhibits enzyme uptake in human MPS I fibroblasts by interfering with M6PR-mediated uptake⁹. Cell uptake measuring intracellular IDUA activity was compared in cells incubated with mammalian cell-derived (mcd-) IDUA or IDUA:RTB with and without pre-incubation with neutralizing canine serum. Consistent with previous reports⁹, serum from high anti-IDUA titer dogs inhibited uptake of mcd-IDUA (>90%). In contrast, more than 90% of IDUA:RTB was successfully taken up into cells (<10% uptake inhibition) even in the presence of high-titer anti-IDUA antibodies.

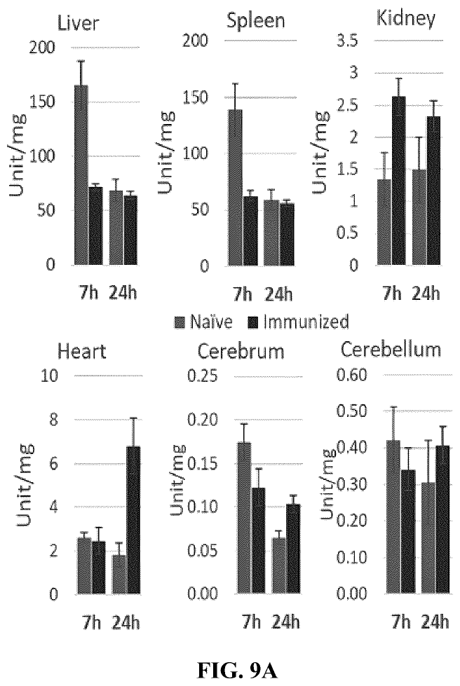

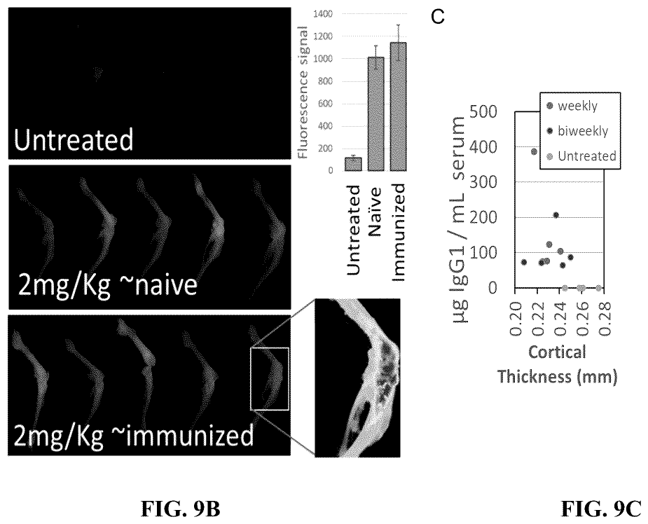

The impact of ADA was also tested in vivo (FIG. 9). A protocol was developed to induce high-titer neutralizing antibodies using med-IDUA immunizations in KO mice. Naïve and immunized mice were subsequently treated with a single dose of fluorescent-labeled IDUA:RTB^{IRDye800} (2 mg/Kg) and organs were harvested at 7 or 24 hr after infusion (n=5 per timepoint) and analyzed for IDUA enzyme activity. Contrary to what has been reported using CHO-derived IDUA, the present analyses with IDUA:RTB did not show the signature ADA-mediated uptake reduction in kidney and heart⁴². In contrast, an increase in activity was seen in these organs (FIG. 9A). The amount of enzyme detected in the CNS was not correlated with ADA titers (FIG. 9A), suggesting that RTB maintained delivery to hard-to-treat sites even in the presence of anti-IDUA antibodies. The fluorescent intensity in legs (femur and tibia) was analyzed on samples collected 7 h after infusion using LiCOR Pearl bioimager. Control mice were injected with equivalent mols of unreactive dye (IRDye800CW carboxylate). As shown in FIG. 9B, the fluorescent signal detected in bone was not significantly different, suggesting that ADA does not impact enzyme delivery to skeletal tissue. A more detailed analysis of the bone samples showed that most of the fluorescence signal within the bone was detected near the growth plate of the tibia and femur which support the ct-microarchitecture results seeing during bone growth and remodeling (FIGS. 6B and 7).

In the long-term administration experiment (FIG. 7), after quantifying the ADA levels in serum, the impact of antibody levels on therapeutic responses was studied; no effect of frequency (weekly vs. biweekly) on antibody levels was found using an equivalent dose (1 mg/kg). Most importantly, no correlation between serum antibody levels and a negative therapeutic effect on the bone was found (FIG. 9C). In this correlation analysis, some of the mice with higher antibody titers achieved complete normalization of cortical thickness, suggesting that the development of ADA does not hinder delivery of the enzyme to the bone and its therapeutic effect.

The dramatic improvement of skeletal pathologies shown in the MPS I model, establishes a platform for treatment of other metabolic disorders with musculoskeletal pathologies such as Morquio A, a disease exemplified by its extreme skeletal manifestation and without effective strategies to address these pathologies.

### Example 8-RTB:GALNS for Morquio A

### ID of Fully Functional RTB:GALNS Fusion.

RTB lectin fusions with the GALNS enzyme were produced and characterized. Constructs displaying different fusion orientation and codon usage were tested for yield and activity to obtain our lead candidate. Inventors expressed the human GALNS sulfatase (corrective enzyme for MPS IVA) and optimized a SUMF1 co-expression system enabling the production of highly active sulfatases in a plant bioproduction system. The SUMF1 co-expression platform supports highly efficient conversion of the active site cysteine to formylglycine required for sulfatase enzymatic activity. The results demonstrate that RTB:GALNS produced in SUMF1 transgenic plants yield a full-length product that retains RTB lectin selectivity and GALNS sulfatase activity, indicating modification to fully active forms.

### Product Purification.

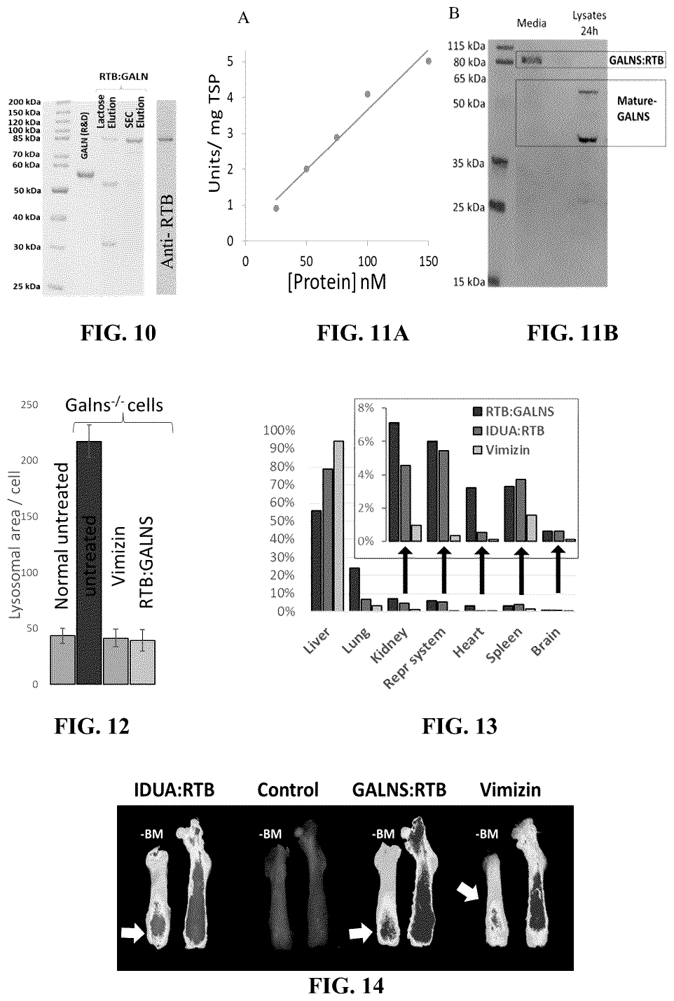

RTB selectively binds to lactose resin (lactose linked to agarose), providing a key affinity capture and concentration step. Lactose affinity chromatography is scalable and will remain our primary purification step. A simple 3-step purification protocol has been developed for RTB fusions that typically yields product at >95% purity. This process involves homogenization of leaf material in extraction buffer, ammonium sulfate precipitation (AmSO₄ ppt), lactose affinity chromatography, and a size exclusion column as a polishing step (Superdex 200). RTB:GALNS was purified using these standard protocols, obtaining enzymatically active fusion product with high levels of purity (>98%) (FIG. 10) with protein identity confirmed at each step by Western blot using anti-RTB antibodies.

### RTB:GALNS Shows Efficient Cell Uptake and Lysosomal Delivery.

RTB-mediated uptake kinetics and intra-cellular processing are key components of the distinct in vivo biodistribution and therapeutic response of the subject technology. RTB's absorptive-mediated uptake is not limited by abundance or cycling kinetics associated with specific surface receptors and does not compete with receptor ligands. RTB:GALNS was therefore tested for uptake into Morquio A patient fibroblasts (galns^{-/-}; Coriell Institute GM00958). Morquio fibroblasts were incubated with different concentrations of enzyme in media, harvested at 24 h by trypsinization, lysed, and assayed for intracellular GALNS activity. A dose-dependent increase in intracellular activity was seen (FIG. 11A), showing no saturation. This data suggest that the RTB:GALNS fusion protein behaves similarly to other RTB fusion products^{48,49} providing a highly efficient enzyme uptake into cells.

Lysosomal enzymes are typically proteolytically processed to mature forms within the lysosome to obtain optimal catalytic activity and stability. In all RTB fusions previously tested, once the full-length product reaches the lysosomes, RTB is degraded, and the human enzymes are processed into their native mature forms^{15,48,50,51}. The human GALNS precursor has a molecular weight of 60 kDa; in human tissues GALNS is present as an oligomer consisting of polypeptides of 40 and 15 kDa. In a pulse-chase study using Morquio A patient fibroblasts (galns^{-/-}), the lysates of cells treated with RTB:GALNS were analyzed by anti-GALNS western blots. After 24 h of incubation, the full-length fusion product (87 kDa) was not detected in lysates. Only the 60 kDa and 40 kDa mature forms were detected intracellularly, suggesting correct delivery and proteolytic activation of the enzyme within the lysosomes (FIG. 11B).

### RTB:GALNS Corrects Lysosomal Disease in Morquio a Human Cells.

Efficacy studies were performed using human patient (GALNS^{-/-}) fibroblasts. Morquio A fibroblasts display enlarged lysosomes compared to normal fibroblasts due to excessive accumulation of sulfated GAG substrates within this organelle. These phenotypical differences can be detected by staining of lysosomes with Lyso Tracker®^{52,53}. The therapeutic response of RTB:GALNS was compared to Vimizin, the FDA-approved recombinant GALNS protein using a high-throughput automated fluorescence imaging system (BioTek Lionheart), which analyzes thousands of cells per treatment group and quantifies therapeutic responses with statistical power. Normal and Morquio A patient fibroblasts (GALNS^{-/-}) were cultured in a 96-well plate in presence of 200 μg/mL of chondroitin sulfate. After attachment, a set of wells containing Morquio A cells were left untreated or were treated with 500 ng/ml of GALNS equivalents of Vimizin or RTB:GALNS. After 24 h, wells were scanned and analyzed for lysosomal area per cell based on Lysotracker signal. Cells were DAPI-counterstained to normalize lysotracker signal by cell count. As shown in FIG. 12, RTB:GALNS normalized the lysosomal phenotype as well as Vimizin, demonstrating delivery of the enzyme to the substrate accumulation site and bioactivity once the enzyme reaches lysosomes.

RTB:GALNS Biodistribution in Mice is Similar to IDUA:RTB and Different from Vimizin.

RTB-mediated biodistribution in RTB:GALNS was tested by intravenous administration of fluorescent labelled products (Dylight⁶⁸⁰) in mice. Ex-vivo fluorescence intensity was used to compare organ biodistribution from mice treated with RTB:GALNS or IDUA:RTB. Biodistribution of the fluorescent labelled commercial drug Vimizin® was also tested as a positive control. Normal C57BL/6J mice were subject to a chlorophyll-free dict for a week to prevent autofluorescence in organs. Eight weeks old mice were treated with 2 mg/kg of RTB:GALNS⁶⁸⁰, IDUA:RTB⁶⁸⁰ or Vimizin⁶⁸⁰ and harvested 24 h after infusion (IDUA:RTB serum half-life is 12 min).

Mice were perfused and organs were scanned using a LiCor Pearl imaging system. Fluorescence intensity in each organ was quantified and expressed as a percentage of total fluorescence detected in all organs. The overall biodistribution pattern of the two RTB-fused products is very similar. Higher signal detected in other organs than liver indicates that RTB is the predominant driver for organ biodistribution (FIG. 13). In contrast, Vimizin biodistribution is mediated by the attachment of phosphorylated glycans to the M6P receptors in cell surface. Using this uptake mechanism, >90% of the administered enzyme ends up in the liver, preventing it from being distributed to other critical organs at larger proportions. Previous tests by the inventors with fluorescence-labeled med-IDUA (Aldurazyme equivalent/commercial ERT drug) showed analogous liver predominance (>90%).

The biodistribution pattern in bone was analyzed. Femurs were dissected and imaged. Fluorescent intensity was detected within the bone in all treatments (FIG. 14) with a pattern suggesting significant localization to the bone marrow (BM). To further assess fluorescence was coming from bone tissue, proximal epiphysis of the left femur was excised, and bone marrow was removed by high-speed centrifugation (−BM). After bone marrow removal, fluorescence distribution differs in RTB-fused products; the highest fluorescence intensity for RTB products was localized near the growth plate of the femur, while highest intensity in the Vimizin treated mice was predominantly localized in the diaphysis of the bone. (FIG. 14; white arrows). Delivery of enzyme to the proliferative zone of the growth plate is key for therapeutic response of the replacement enzyme. Due to the avascular nature of cartilage, efficient delivery of the enzyme to cells that are embedded in this region remains challenging^{54,55}. However, RTB-enzyme product corrects the bone pathology in MPS I mice, confirming delivery and therapeutic efficacy in the critical cells that regulate bone growth and remodeling, and that are adversely affected by GAG accumulation.

Short-term pharmacokinetics of lectin-delivered RTB:GALNS is assessed in the Morquio A (Galns^{-/-}) mouse model. The Morquio A mouse model was developed by targeted disruption of the Galns gene in embryonic stem cells to generate a knock-out mouse model (Galns^{-/-}). In this model, bones display aberrant growth and accumulation of storage material. GAG accumulation is also detected in cells and tissues relevant for Morquio A patients, including heart valves, trachea and chondrocytes in growth plate, articular cartilage, ligaments, and meniscus.

Clear histomorphometry and pathological differences are detected in the growth plate of KO mice when compared to normal mice. Growth plates are irregular and chondrocytes display excessive accumulation of storage material. This knockout mouse model is adequate for evaluation of treatment and correction of cells and tissues responsible for the aberrant bone growth such as chondrocytes of the growth plate. This model also mirrors the anti-drug antibody response seen in most Morquio A patients treated with the current FDA-approved drug. The complete lack of GALNS activity in tissues facilitates precise quantification of the delivered recombinant enzyme in various tissues.

RTB:GALNS pharmacokinetics and biodistribution is assess in mixed gender Galns^{-/-} mice (n=9) at 12 weeks old when bones are fully developed, mice are treated with 6 mg/kg of GALNS equivalents and bled at 1, 3, 5, 15, 30, 45 min after infusions. A subset of mice is harvested at 0.5, 1, and 3 h after infusion to track enzyme processing and maturation of liver tissues by western blot. Another set of nine mice (n=9) are treated using the same dosage and harvested at 6, 24, and 48 h after injection. Only terminal plasma is collected in this group. Enzyme activity levels are measured in liver, spleen, kidney, heart, lung, trachea, bone, and bone marrow. GALNS enzymatic activity in plasma and tissues are measured using 4-methylumbelliferyl-b-galactopyranoside-6-sulfate fluorometric assay as described previously. Enzyme delivered to liver tissue is visualized by Western blot for correct processing and maturation of the product. After reaching the lysosomes, RTB is typically proteolytically cleaved, and the GALNS enzyme should be processed from the 60 kDa precursor to polypeptides of 40 and 15 kDa. Maturation of GALNS within the mouse tissue is tracked in liver lysates collected at 30 min. and 1, 3, 6 and 24 h after infusion and detected by western blot using anti-GALNS antibodies and anti-RTB antibodies as described.

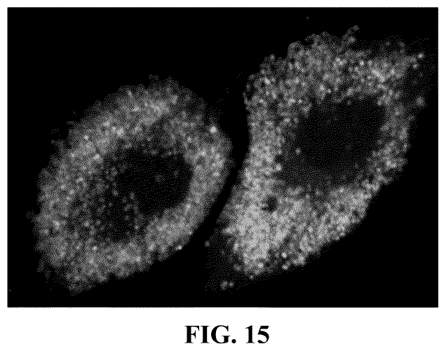

Uptake of fluorescence-labeled RTB:GALNS into chondrocytes, cartilage and heart valves. Delivery of therapeutic enzymes to growth plate chondrocytes and heart valves is challenging due to the avascular nature of these tissues. KS accumulates in the heart valve of MPS IVA patients and represents an important cause of morbidity in this disease. RTB:GALNS and Vimizin products are fluorescently labelled with Dylight680. The delivery of labeled product is assessed in vitro and in vivo. Human chondrocytes (C20A4, Sigma #SCC041) are grown in glass-bottom 35 mm plates and treated with 0.5 μg/mL of fluorescent-labelled RTB:GALNS or mcd-GALNS. Cells are stained with Lysotracker® red (594) and fixed at different timepoints for co-localization analysis. Cell uptake of RTB:eGFP fusion was tested in chondrocyte primary cultures from Morquio A patients. After 1 h incubation with 0.5 μg/mL of protein, fluorescence was detected intracellularly in vesicles (FIG. 15). This pattern is consistent with RTB endocytosis and delivery to lysosomes seen with other human cell types (e.g., Caco2, fibroblasts, Hela cells).

Delivery to growth plate and articular cartilage in vivo are tested using Galns^{-/-} mice. Fluorescent-labeled RTB:GALNS is administered by tail-vein injection using 1, 2, and 4 mg GALNS-equivalents per kg. Femurs and hearts are excised 6, 24, or 48 hr after the infusion of each dose tested. Tissues are fixed, bones decalcified, paraffin-embedded and sectioned at 5 uM. Sections are immuno-stained with LAMP2 and scanned by fluorescent microscopy.

RTB:GALNS is broadly distributed, is appropriately processed in lysosomes, and, most critically, is localized in bone and connective tissues.

### Example 9-Evaluate the Impact of Dosing and Treatment Initiation after Long-Term Administration of RTB:GALNS

The goal is to define critical components of drug dosage and timing of treatment initiation to obtain optimal disease correction in the Morquio A mouse model and meet FDA's nonclinical program recommendations. The experimental design is based on previous ERT studies to facilitate comparisons with the existing ERT strategy (e.g., CHO-derived Vimizin). ERT with Vimizin administered to adult Morquio A mice provides a slight pathological improvement in ligaments and connective tissues surrounding the articular cartilage after 12 weekly infusions of 250 U/g (˜1 mg/kg). The growth plate region also shows limited pathological improvements in these studies even at higher doses (4 mg/kg), confirming that complete remission of bone lesions, especially in the avascular cartilage tissue, remains an unmet challenge in the mouse model as also observed in Morquio A children.

The data in animal studies using IDUA:RTB demonstrate that the lectin delivery technology corrects bone pathology using weekly doses of the replacement enzyme at 2 mg/kg treatment when started in either young or adult mice indicating that a therapeutic response is achieved even during the bone remodeling phase. Morquio A patients suffer from early deleterious effects of GAG accumulation in the growth plate that may result in irreversible damage and, consequently, abnormal bone growth despite later therapeutic interventions. The first months of life, therefore, represent the best opportunity to prevent bone deformities. Morquio A mouse model differs from the MPS I model with respect to the type and amount of GAG storage material observed.

Vimizin is provided to patients at a dose of 2 mg/kg, and this dose (2 mg GALNS equivalents/kg) will be used as baseline. One higher (6.0 mg/kg) and one lower (1 mg/kg) dose will be used to compare efficacy. Efficacy assessments are based on key readouts established for this mouse model. In addition to skeletal pathology and correction, potential ADA responses are assessed in these mice as anti-GALNS antibodies have been an issue for both the mouse model and patients with long-term Vimizim treatment. The impacts of antigen-antibody interactions on in vivo biodistribution are complex, affecting serum half-life, the rate and selectivity of cell/tissue/organ uptake, and intracellular processing and stability of the enzyme.

Mouse trial design RTB:GALNS dose evaluation and age of treatment initiation. Groups of male Ganls^{-/-} mice (n=10) are randomly assigned to treatment cohorts. As observed in MPS I mice, micro-architecture phenotypical differences of Morquio A KO mice are more severe in males (unpublished data). Unaffected WT siblings are assigned to a cohort as controls,

To test the therapeutic effect of RTB:GALNS during bone remodeling, eight-week-old mice are treated weekly by tail vein injection at the doses indicated (expressed as GALNS equivalents). A cohort using the FDA-approved dose of Vimizin (CHO GALNS) is carried in parallel as a control. Mouse studies using Vimizin conclude that early administration improved the efficacy of the drug. Parallel cohorts are carried out starting with newborn mice (day 2-3) injected through a superficial temporal vein. Mice showing signs of adverse reactions are injected with dexamethasone (2 mg/ml; 10 μl) 1 h before subsequent RTB:GALNS i.v. treatment and carefully monitored post-injection. Blood samples are collected at baseline, and at subsequent 4-week intervals, for measuring plasma KS and anti-GALNS antibody responses. Mice are harvested 24 h after the last dose (16 weeks of age). At the endpoint, terminal blood is collected, and tissues are dissected with each organ/tissue processed 1) for biochemical analyses (enzyme activity; GAG levels) or 2) fixed for histology and 3) for CT scanning. Harvested tissues include liver, spleen, kidney, heart, lung, brain, trachea, bone, and bone marrow.

Although, skeletal deformities are the most evident pathological feature of the disease, systemic accumulation of GAG in MPS IV patients leads to long term neurological, cardiovascular, and respiratory complications. Storage material is analyzed in tissues from brain, heart, liver, spleen, lung, bone, and trachea. Mono-sulfated KS levels in serum and tissues are quantified using liquid chromatography-tandem mass spectrometry as described previously. Enzyme activities are measured in liver, spleen, kidney, heart, lung, femur, eye, trachea and bone marrow.

Treatments for the progressive skeletal disease manifestations associated with many lysosomal diseases remain lacking or ineffective. The RTB lectin shows surprising efficacy in targeting cells of the bone and connective tissue and has been shown to significantly improve skeletal pathologies in a model system (MPS I). The present invention mobilizes this discovery to develop an ERT for Morquio A capable of blocking skeletal disease progression and improving the mobility and quality of life for these patients.

### Example 10-Fusion Comprising GAA and RTB for Pompe Disease

Pompe disease (OMIM: 232300, also known as Glycogenosis type II) is an autosomal recessive metabolic disorder caused by genetic deficiencies of GAA, the sole enzyme catabolizing glycogen to glucose in lysosomes. GAA deficiency results in lysosomal accumulation of glycogen, leading to progressive disruption of cellular function, especially in smooth, cardiac, and skeletal muscle cells. To date, ERT with recombinant human GAA synthesized in CHO cells (alglucosidase alfa; marketed as Myozyme or Lumizyme, Sanofi Genzyme) is the only approved treatment for Pompe disease and is administered intravenously at doses of 20 mg/kg body weight. Although it has provided substantially prolonged survival in many IOPD patients, predominately by reducing cardiac hypertrophy and preventing cardiac failure, current ERT has several limitations including 1) poor targeting to one of the main affected tissues, skeletal muscle; 2) anti-drug antibody (ADA) immune responses to ERT drug (particularly harmful for CRIM-patients that totally lack GAA), and 3) the inability to cross the blood brain barrier to treat CNS pathologies.

Pompe disease is a devastating rare muscle disease. In its most severe form, infants have severe respiratory/cardiac complications that often lead to death within the first year. Late-onset patients suffer from progressive muscle weakness leading to loss of motor function and serious breathing problems, with pulmonary failure due to diaphragmatic weakness as a typical cause of death. Pompe is caused by deficiencies in acid alpha-glucosidase (GAA), the sole enzyme responsible for lysosomal glycogen degradation, resulting in pathogenic glycogen accumulation—especially in lysosomes of smooth, cardiac, and skeletal muscles cells, although other organs such as the brain are affected. GAA Enzyme Replacement Therapy (Alglucosidase alfa; mammalian cell-derived or mcd-GAA) is the only approved Pompe treatment. This ERT slows disease progression but does not halt or reverse the disease due primarily to insufficient access to critically involved muscle tissues. Drug efficacy is further compromised by development of immune responses to the ERT in many patients making Pompe the prototypical disease for anti-drug antibody (ADA) response to biologic drugs.

RTB, a plant galactose/galactosamine-binding lectin, was developed as a unique enzyme carrier for genetically fused human lysosomal enzymes. RTB has a remarkable ability to deliver fused enzymes to lysosomes of the classic “hard-to-treat” cells, tissues and organs including skeletal muscle, heart and the central nervous system critical for treating Pompe disease.

RTB directs uptake based on lectin-mediated binding to cell surface glycoproteins and glycolipids using absorptive-mediated mechanisms in contrast to receptor-mediated uptake used by GAA-based ERT and all other current ERTs on the market for lysosomal diseases. Thus, RTB endo/transcytosis is not limited by receptor abundance or cycling kinetics and appears capable of mobilizing associated enzymes into all cell types and tissues tested to date—including crossing the blood-brain barrier to treat the CNS and accessing myocytes of skeletal muscle.

The results demonstrated that RTB could deliver fused enzyme into cells and hard-to-treat tissues even in the presence of neutralizing anti-enzyme antisera in vitro or in vivo in mice displaying high-titer immune responses to the ERT enzyme. Thus, the RTB carrier module has considerable potential to address the most significant limitations and unmet medical needs for Pompe ERT treatment: efficient delivery to muscle cells, mitigation of ADA immune responses, and delivery across the blood-brain-barrier to treat the CNS.

### Product Production and Purification.

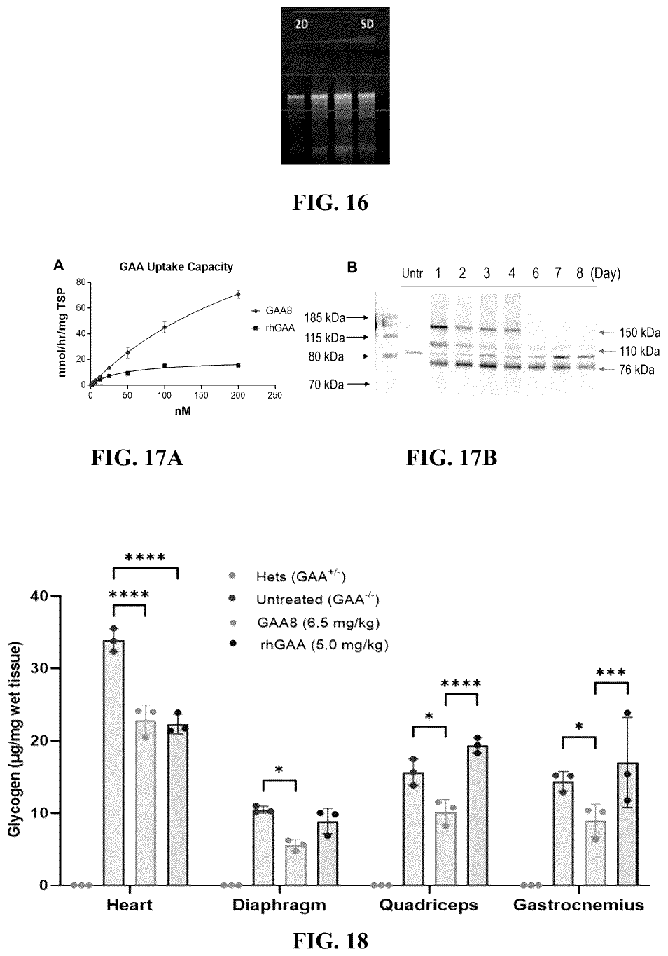

GAA:RTB fusion proteins was produced and characterized in N. benthamiana. Fusion cassettes were introduced into a modified pBIB-Kan plant expression vector downstream of a de35S constitutive promoter, TEV translational enhancer, and the plant-optimized signal peptide (PoSP) were transformed into Agrobacterium tumefaciens strain LBA4404 (Agro). Agro cultures carrying the various constructs were infiltrated into leaves of intact 4-week-old N. benthamiana plants by vacuum infiltration and leaves were harvested 4 days after infiltration. Western blot analysis using anti-RTB antibody showed expression of full-length GAA-RTB fusion proteins from 2 to 5 days after the transfection (˜145 kDa), as shown in FIG. 16.

### GAA:RTB Shows Efficient Cell Uptake and Lysosomal Delivery.

To determine whether the RTB lectin would effectively mediate cell uptake, Pompe fibroblasts (Coriell GM00248) derived from an infantile-onset patient were treated with various concentrations (1.6-200 nM) of purified GAA8 or rhGAA for 4 hours to allow cellular uptake. After refreshing culture media, cells were incubated overnight and harvested after 24 hours. Cell lysates were used for enzyme assay. Cells treated with GAA8 showed higher protein uptake in terms of enzyme activity as well as higher saturation level compared to cells treated with rhGAA (FIG. 17A). To further analyze GAA8 proteolytic processing within fibroblasts, Pompe fibroblasts treated with 50 nM of GAA8 were harvested daily for up to 8 days. Cells lysates were analyzed by western blot detected with anti-GAA antibodies (ab137068, Abcam). As shown in FIG. 17B, the 76 kDa “activated/maturated” GAA products is still present after 8 days incubation suggesting significant stability of the processed form.

### Example 11-Evaluate the Impact of Dosing and Treatment Initiation after Long-Term Administration of GAA:RTB

The GAA:RTB product distributes to key cells and organs, especially skeletal muscle, heart in the Pompe mouse model. The KO Pompe 6neo/6neo mouse strain was used, which has been used to test multiple GAA variants for tissue distribution and glycogen reduction. B6; 129-Gaatm 1 Rabn/J mice (Jackson Laboratory) exhibiting a progressive accumulation of lysosomal glycogen in heart and skeletal muscle. In this model, Alglucosidase alfa (20 mg/kg) showed poor targeting to skeletal muscle.

4-week-injections of GAA8 were performed and the reduction of glycogen was measured in Pompe mice to demonstrate whether RTB-lectin would deliver GAA to the key organs and tissues in vivo. At age of 8 weeks, Pompe mice (n=3) received weekly tail vein injection of either GAA:RTB at 6.5 mg/kg (equivalent to 5.0 mg/kg of rhGAA) or rhGAA at 5 mg/kg. Aged matched untreated Pompe mice (n=3) and heterogeneous mice (n=3) were used as controls. After 4-weck treatment, all mice were euthanized and perfused prior to harvest. The glycogen levels of signature skeletal muscle (gastrocnemius and quadriceps), smooth muscle (diaphragm), heart, and CNS tissues (cortex, cerebellum, and spinal cord) were quantified by a commercial glycogen assay kit (BioVision). As shown in FIG. 18, both rhGAA and GAA:RTB have a similar glycogen reduction in heart but remarkably, GAA:RTB exhibited a superior efficacy in diaphragm, gastrocnemius, and quadriceps tissues, conferment efficient delivery and correction of the therapeutic product to the musculoskeletal tissue.

### Example 12-RTB Delivers Collagen-Modifying Enzymes to the ER of OI Type VII Osteoblasts and Fibroblasts

RTB was operatively associated with two different enzymes involved in collagen modification by recombinant production of genetic fusions. These included RTB fused to human prolyl-3-hydroxylase 1 (P3H1) yielding P3H1:RTB (termed P3) and fused to the human scaffolding protein cartilage associated protein (CRTAP) yielding CRTAP:RTB (termed C3). Primary fibroblast and osteogenic cell cultures were established from newborn pups having either CRTAP−/− (OI VII mouse) or CRTAP+/− (normal Het sibling) genotypes. Following several passages, near confluent cultures were treated with P1, C1, C3, P3 or C3/P1 complex at several doses and tested for uptake and localization at 1, 4, and 24 hours.

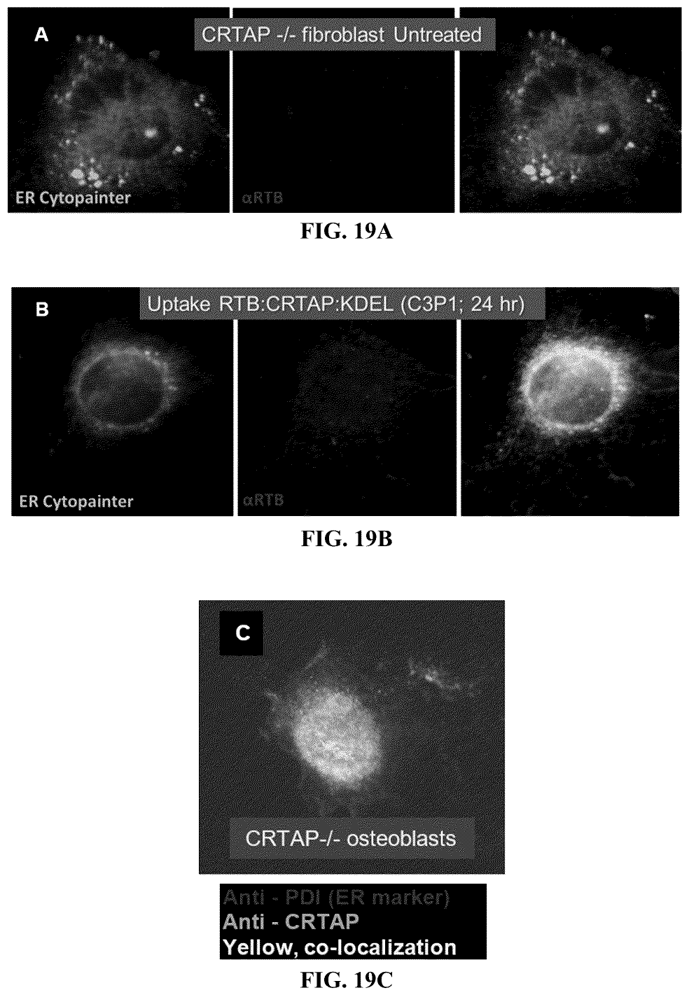

Fluorescent imaging was performed using 1) DAPI to stain nuclei, 2) ER Cytopainter (FIGS. 19A and 19B) or anti-PDI (FIG. 19C) as ER markers, and 3) antibodies directed against RTB or CRTAP to detect the plant-made products. Although P1 and C1 (products lacking the RTB lectin) did not enter cells, the C3/P1 complex (shown in FIG. 19B) was efficiently taken up into CRTAP−/− cells (both fibroblast and osteoblast cultures established from calvarial explants from OI-VII mice) indicating that the RTB lectin supported adsorptive-mediated endocytosis of the complex.

In addition, the product co-localized with ER markers indicating effective trafficking to the ER site of collagen processing. This is significant as RTB normally traffics primarily to the lysosomes. To counter this, all of the constructs were engineered to include an N-terminal KDEL “ER Retrival” sequence. These experiments demonstrate that, in addition to RTB-mediated cell uptake, the RTBER strategy correctly placed the product in the key organelle critical for collagen modifications by P3H1 and CRTAP.

### Example 13-RTB Delivers Corrective Enzyme to Reduce Bone Pathology in Mouse Models of Osteogenesis Imperfecta (OI)

Rare recessive forms of OI typically show skeletal malformation as neonates. Corrective replacement proteins need to be delivered to skeletal growth plates and cartilaginous tissues as early as possible to address disease progression. Additionally, they need to get into these cells and accumulate within the ER and assemble into metabolically functional collagen-processing complexes. As described in Example 12, RTB fused with either CRTAP or P3H1 meets these criteria.

To assess functionality in vivo, OI type VII mice (CRTAP−/−) and OI type VIII mice (P3H1−/−) are treated with CRTAP:RTB, P3H1:RTB or CRTAP:RTB+P3H1 complex as appropriate for their genetic deficiency. Treatment is initiated at week 2 via IP administration testing doses ranging from 1 to 5 mg/kg. Mice are treated weekly with administration route shifting to IV by age 5-6 weeks old. Mice are analyzed by the tests described in Example 4 with the exception that no GAG analyses are performed (GAG is MSP specific disease substrate). Treated mice are compared to untreated OI mice and normal (WT or heterozygous siblings). Treated mice show significant improvement in bone structure and formation, mobility, skin flexicity, and respiratory function (indicator of cartilage function).

### Example 14-RTB Delivers Active β-Galactosidase Enzyme to Bone, Cartilage, and Connective Tissues to Address Skeletal Pathologies in Morquio B Disease

Morquio B Disease (MBD; MPS IVB) is a rare autosomal recessive lysosomal storage disease, presenting with a peculiar type of skeletal dysostosis multiplex, that is similar to that observed in GALNS-related Morquio A disease. MBD (also known as Mucopolysaccharidosis IVB) is caused by specific allelic variants of the GLB1 gene, which encodes the lysosomal hydrolase beta-Galactosidase (β-Gal). β-Gal deficiencies are typically associated with a progressive neuronopathic condition, GM1 gangliosidosis, spanning from infantile to juvenile and late onset forms.

In contrast to GM1 gangliosidosis patients, MBD presents with a unique dysostosis multiplex, causing a peculiar type of spondyloepiphyseal dysplasia involving trabecular parts of long bones and the spine. MBD may present as pure skeletal phenotype (pure MBD) or in combination with the neuronopathic manifestations seen in type 2 (juvenile) or type 3 (late onset) GM1 gangliosidosis (MBD plus). Whereas GM1 gangliosidosis involves pathogenic accumulation of the β-Gal substrates, GM1 and GA1 gangliosides, primarily in the CNS, accumulation of keratan sulfate (also a substrate for the lysosomal β-Gal enzyme) in bones and cartilage is the dominant factor leading to MBD.

Knock-out mouse models targeting the GLB1 gene recapitulate the progressive neurodegeneration observed in early-onset GM1 gangliosidosis patients. However, the skeletal defects associated with MBD are not observed, even with CRSPR-Cas 9 approaches to reproduce the specific MBD allelic variants of GLB1 gene. Thus, an appropriate mouse model is not available to test efficacy of β-Gal:RTB fusions in correcting the skeletal phenotypes associated with MBD. However, correction of the GM1 gangliosidosis neurological disease and biodistribution to bone and other critical tissues and cells of the skeletal system can be demonstrated for β-Gal:RTB using the currently available β-Gal-KO mouse model.

Biodistribution to bones and cartilaginous cells and tissues is shown by administering (tail vein injection) fluorescently labeled β-Gal:RTB fusion (e.g., with Dylight) and assessing fluorescence via whole animal imaging and ex-vivo delivery to bone, connective tissue, and cartilage (e.g. trachea) as shown for the IDUA:RTB fusion for the MPS I mouse and for RTB:GALNS fusion in the Morquio A model.

β-Gal:RTB product can improve bone growth and correct associated skeletal system pathologies in patients with Morquio B.

### Example 15-RTB Delivers Corrective Enzyme to Reduce Bone Pathology in Maroteauz-Lamy Syndrome (MPS VI)

Maroteaux-Lamy syndrome (also known as Mucopolysaccharidosis VI; MPS VI) is a rare autosomal lysosomal disease caused by deficiencies in the N-acetylgalactosemine-4-sulfatase (arylsulfatase B; ARSB). The disease presents with progressive and severe skeletal. SUMF1^{KDEL} transgenic plants is used to produce the fusion protein comprising RTB lectin and a human sulfatase (e.g., ARSB), which retains RTB lectin binding selectivity and sulfatase enzymatic activity.

The sequence encoding ARSB (˜60 kDa; NCBI J05225.1) can be synthesized (GeneArt) based on native (nat) and tobacco-codon optimized (opt) DNA sequences. Fusion cassettes produced using “In-Fusion” (Clontech) can be sequence confirmed, introduced into a modified pBIB-Kan plant expression vector (de35S promoter; TEV translational enhancer and our plant-optimized signal peptide-PoSP), and plasmids introduced into Agrobacterium tumefaciens strain LBA4404. Agro cultures carrying the constructs (including pBIB-Kan “empty vector” control) can be infiltrated into leaves of intact 4-weck old N. benthamiana plants by vacuum infiltration. Optimal harvest times are assessed by comparing yields in crude extracts of plants harvested at 48, 72, 96 and 120 h post-infiltration. Initial readouts are based on sulfatase activity per gFW leaf and TSP in crude extract.

To optimize extraction conditions, infiltrated leaf material can be homogenized using the standard test buffer panel (varies pH, ionic strength, buffering compounds) and analyzed by a rapid and simple “dual-activity” 96-well plate assay. This assay entails lectin binding to immobilized glycoprotein (asialofetuin) and quantification of bound sulfatases-RTB fusion by sulfatase activity assay specific for ARSB. Yields of full-length product can be assessed by western immunoblot using antibodies against RTB (BioStrategies LC) and ARSB (AF4415, R&D).

For fusion protein purification, an ammonium sulfate precipitation is used followed by lectin-affinity chromatography (lactose resin) and size-exclusion chromatography for product recovery at 10-15% at >95% purity. Products are monitored/quantified during purification by using a “lectin-selective” ELISA and sulfatase assay. Purity of the protein can be assessed by SDS-PAGE stained with SimplyBlue™.

To demonstrate RTB-mediated delivery of enzymatically active sulfatases into cells and correction of cellular disease phenotype, uptake capacity of the sulfatase:RTB fusion and mcd-sulfatase is compared using MPS VI deficient fibroblasts (GM00519). Maximum uptake capacity can be determined by measuring intracellular sulfatase activity in cells after incubation with different concentrations of the protein in media. This analysis determines the enzyme concentration required to reach uptake saturation in cells. As with most lysosomal hydrolases, the sulfatases is proteolytically processed in lysosomes to mature forms i.e., ARSB (˜58 kDa zymogen->˜43, 7 and 8 kDa. For RTB fusions, the RTB domain is usually cleaved as well. Protein processing in the sulfatase: RTB added to the fibroblast cultures can be assessed by Western blots to further confirm lysosomal delivery.

To assess drug efficacy in vitro, GAG substrate and lysosome volume reduction using lysotracker can be assessed on treated sulfatase^{-/-} fibroblasts using a high-through-put cell imaging system (Lionheart LX, BioTek). Briefly, fibroblasts from normal individuals (Coriell #GM00010) and MPS VI patients are cultured in a 96-well format to near confluency. A set of diseased fibroblasts is incubated for 24 hr with 1.6-200 nM med-sulfatases ARSB (4415-SU) from R&D systems or the sulfatase:RTB fusions, fixed, fluorescence stained and counterstained with DAPI. Lysosomal signal can be quantified, normalized by cell count and compared to untreated controls using Gen5 software.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 4,938,949
U.S. Pat. No. 5,034,322
U.S. Pat. No. 5,106,739
U.S. Pat. No. 5,589,610
U.S. Pat. No. 5,625,136
U.S. Pat. No. 5,639,948
U.S. Pat. No. 5,661,017
U.S. Pat. No. 5,705,484
U.S. Pat. No. 6,455,760
U.S. Pat. No. 6,462,185
U.S. Pat. No. 6,696,623
U.S. Pat. No. 6,884,419
U.S. Pat. No. 7,011,972
U.S. Pat. No. 7,410,779
U.S. Pat. No. 7,867,972
U.S. Pat. No. 7,964,377
U.S. Published application No. 20030084486
U.S. Published application No. 20030177536
U.S. Published application No. 20040019934
U.S. Published application No. 20040067506
U.S. Published application No. 20040078841
U.S. Published application No. 20040123349
European Application No. EP1528104
1. Montaño A M, Tomatsu S, Gottesman G S, Smith M, Orii T. International Morquio A Registry: Clinical manifestation and natural course of Morquio A disease. J Inherit Metab Dis. 2007; 30(2):165-174.
2. Puckett Y, Mulinder H, Montaño A M. Enzyme Replacement Therapy with Elosulfase alfa for Mucopolysaccharidosis IVA (Morquio A Syndrome): Milestones and Challenges. Expert Opin Orphan Drugs. 2017; 5(9):741-752.
3. Davison J E, Kearney S, Horton J, Foster K, Peet A C, Hendriksz C J. Intellectual and neurological functioning in Morquio syndrome (MPS IVa). J Inherit Metab Dis. 2013; 36(2):323-328.
4. Tomatsu S, Sawamoto K, Alméciga-Díaz C J, et al. Impact of enzyme replacement therapy and hematopoietic stem cell transplantation in patients with morquio a syndrome. Drug Des Devel Ther. 2015; 9:1937-1953.
5. Tomatsu S, Alméciga-Díaz CJ, Montaño A M, et al. Therapies for the bone in mucopolysaccharidoses. Mol Genet Metab. 2015; 114(2):94-109.
6. Doherty C, Stapleton M, et al. Effect of enzyme replacement therapy on the growth of patients with Morquio A. J Hum Genet. 2019; 64(7):625-635.
7. Urayama A, Grubb J H, Sly W S, Banks W A. Developmentally regulated mannose 6-phosphate receptor-mediated transport of a lysosomal enzyme across the blood-brain barrier. Proc Natl Acad Sci USA. 2004; 101(34):12658-12663.
8. Glaros E N, Turner C T, Parkinson E J, Hopwood J J, Brooks D a. Immune response to enzyme replacement therapy: single epitope control of antigen distribution from circulation. Mol Genet Metab. 2002; 77 (1-2): 127-135.
9. Dickson P, Peinovich M, Mcentee M, et al. Immune tolerance improves the efficacy of enzyme replacement therapy in canine mucopolysaccharidosis I. J Clin Invest. 2008; 118(8):2868-2876.
10. Ponder K P. Immune response hinders therapy for lysosomal storage diseases. J Clin Invest. 2008; 118(8):8-11.
11. Long B, Tompkins T, Decker C, et al. Long-term Immunogenicity of Elosulfase Alfa in the Treatment of Morquio A Syndrome: Results From MOR-005, a Phase III Extension Study. Clin Ther. 2017; 39(1): 118-129.c3.
12. Schweighardt B, Tompkins T, Lau K, et al. Immunogenicity of Elosulfase Alfa, an Enzyme Replacement Therapy in Patients with Morquio A Syndrome: Results from MOR-004, a Phase III Trial. Clin Ther. 2015; 37(5): 1012-1021.c6.
13. Harmatz P R, Mengel E, Geberhiwot T, et al. Impact of elosulfase alfa in patients with morquio A syndrome who have limited ambulation: An open-label, phase 2 study. Am J Med Genet Part A. 2017; 173(2):375-383.
14. Burton B K, Berger K I, Lewis G D, et al. Safety and physiological effects of two different doses of elosulfase alfa in patients with morquio a syndrome: A randomized, double-blind, pilot study. Am J Med Genet Part A. 2015; 167(10):2272-2281.
15. Acosta W L, Ou L, Ayala J, et al. Lectin-mediated delivery of α-L-iduronidase: A novel approach for MPS I enzyme replacement therapy. Mol Genet Metab. 2016; 117 (2):S14-S15.
16. Condori J, Acosta W, Ayala J, et al. Enzyme replacement for GM1-gangliosidosis: Uptake, lysosomal activation, and cellular disease correction using a novel β-galactosidase:RTB lectin fusion. Mol Genet Metab. 2016; 117:199-209.
17. Ou L, Przybilla M J, Koniar B, Whitley C B. RTB lectin-mediated delivery of lysosomal α-1-iduronidase mitigates disease manifestations systemically including the central nervous system. Mol Genet Metab. 2018; 123(2): 105-111.
18. Sandvig K, Bergan J, Kavaliauskiene S, Skotland T. Lipid requirements for entry of protein toxins into cells. Prog Lipid Res. 2014; 54C: 1-13.
19. Olsnes S. The history of ricin, abrin and related toxins. Toxicon. 2004; 44:361-370.
20. O'Hara J, Yermakova A, Mantis N. Immunity to Ricin: Fundamental Insights into Toxin-Antibody Interactions. Curr Top Microbiol Immunol. 2012; 357 (January): 209-241.
21. Sandvig K, van Deurs B. Membrane traffic exploited by protein toxins. Annu Rev Cell Dev Biol. 2002; 18:1-24.
22. Sandvig K, Pust S, Skotland T, van Deurs B. Clathrin-independent endocytosis: mechanisms and function. Curr Opin Cell Biol. 2011; 23:413-420.
23. Sandvig K, van Deurs B. Endocytosis and intracellular transport of ricin: recent discoveries. FEBS Lett. 1999; 452 (1-2):67-70.

24 Simmons B M, Stahlf P D, Russell J H. Mannose Receptor-mediated Uptake of Ricin Toxin and Ricin A Chain by Macrophages. J Biol Chem. 1986; 261(17):7912-7920.
25. Frankel A E, Fu T, Burbage C, et al. Lectin-deficient ricin toxin intoxicates cells bearing the d-mannose receptor. 1997; 300:251-258.
26. van Deurs B, Tønnessen TI, Petersen O W, Sandvig K, Olsnes S. Routing of Internalized Ricin and Ricin Conjugates to the Golgi Complex. J Cell Biol. 1986; 102:37-47.
27. Acosta W, Cramer C L. Targeting Macromolecules to CNS and Other Hard-to-Treat Organs Using Lectin-Mediated Delivery. Int J Mol Sci. 2020; 21(3):971.
28. Simonaro C M, Haskins M E, Schuchman E H. Articular Chondrocytes from Animals with a Dermatan Sulfate Storage Disease Undergo a High Rate of Apoptosis and Release Nitric Oxide and Inflammatory Cytokines: A Possible Mechanism Underlying Degenerative Joint Disease in the Mucopolysaccharidoses. Lab Invest. 2001; 81(9):1319-1328.
29. Clarke L A, Hollak C E M. The clinical spectrum and pathophysiology of skeletal complications in lysosomal storage disorders. Best Pract Res Clin Endocrinol Metab. 2015; 29(2):219-235.
30. Wilson S, Hashamiyan S, Clarke L, et al. Glycosaminoglycan-mediated loss of cathepsin K collagenolytic activity in MPS I contributes to osteoclast and growth plate abnormalities. Am J Pathol. 2009; 175(5):2053-2062.
31. Simonaro C M, D'Angelo M, He X, et al. Mechanism of Glycosaminoglycan-Mediated Bone and Joint Disease. Am J Pathol. 2008; 172(1): 112-122.
32. Kuchn S C, Kochne T, Cornils K, et al. Impaired bone remodeling and its correction by combination therapy in a mouse model of mucopolysaccharidosis-I. Hum Mol Genet. 2015; 24(24):7075-7086.
33. Clarke L A, Wraith J E, Beck M, et al. Long-term Efficacy and Safety of Laronidase in the Treatment of Mucopolysaccharidosis I. Pediatrics. 2008; 123 (1).
34. Pievani A, Azario I, Antolini L, et al. Neonatal bone marrow transplantation prevents bone pathology in a mouse model of mucopolysaccharidosis type I Running title: Skeletal outcome of MPS I mice following neonatal BMT. Blood. 2015; 125(10):1662-1672.
35. Heppner J M, Zaucke F, Clarke L A. Extracellular matrix disruption is an early event in the pathogenesis of skeletal disease in mucopolysaccharidosis I. Mol Genet Metab. 2015; 114(2): 146-155.
36. Pedersen K B, Williams A, Watt J, Ronis M J. Improved method for isolating high-quality RNA from mouse bone with RNAlater at room temperature. Bone Reports. 2019; 11:100211.
37. Yang X, Hatfield J T, Hinze S J, Mu X, Anderson P J, Powell B C. Bone to pick: The importance of evaluating reference genes for RT-qPCR quantification of gene expression in craniosynostosis and bone-related tissues and cells. BMC Res Notes. 2012; 5:222.
38. Azario I, Pievani A, Del Priore F, et al. Neonatal umbilical cord blood transplantation halts skeletal disease progression in the murine model of MPS-I. Sci Rep. 2017; 7 (1).
39. Tomatsu S, Fujii T, Fukushi M, et al. Newborn screening and diagnosis of mucopolysaccharidoses. Mol Genet Metab. 2013; 110 (1-2):42-53.
40. Baldo G, Mayer F Q, Martinelli B Z, et al. Enzyme replacement therapy started at birth improves outcome in difficult-to-treat organs in mucopolysaccharidosis I mice. Mol Genet Metab. 2013; 109(1):33-40.
41. Ou L, Herzog T, Koniar B L, Gunther R, Whitley C B. High-dose enzyme replacement therapy in murine Hurler syndrome. Mol Genet Metab. 2014; 111(2): 116-122.
42. Le S Q, Kan S, Clarke D, et al. A Humoral Immune Response Alters the Distribution of Enzyme Replacement Therapy in Murine Mucopolysaccharidosis Type I. Mol Ther—Methods Clin Dev. 2018; 8:42-51.
43. Radin D, Acosta W. Materials and methods for mitigating immune-sensitization-US patent US20190046637A1. https://patents.google.com/patent/US20190046637A1/en. February 2016.
44. Cohen O, Mechaly A, Sabo T, et al. Characterization and epitope mapping of the polyclonal antibody repertoire elicited by ricin holotoxin-based vaccination. Clin Vaccine Immunol. 2014; 21(11): 1534-1540.
45. Yermakova A, Klokk T I, O'Hara J M, Cole R, Sandvig K, Mantis N J. Neutralizing Monoclonal Antibodies against Disparate Epitopes on Ricin Toxin's Enzymatic Subunit Interfere with Intracellular Toxin Transport. Sci Rep. 2016; 6(1):22721.
46. Yermakova A, Mantis N J. Protective immunity to ricin toxin conferred by antibodies against the toxin's binding subunit (RTB). Vaccine. 2011; 29(45):7925-7935.
47. Montfort W, Villafranca J E, Monzingo A F, et al. The three-dimensional structure of ricin at 2.8 A. J Biol Chem. 1987; 262(2):5398-5403.
48. Ayala J, Acosta W, Condori J, et al. Uptake, lysosomal activation, and disease correction in GM1 Gangliosidosis cells by plant-made β-galactosidase: Lectin fusions. Mol Genet Metab. 2016; 117 (2):S23.
49. Acosta W, Ayala J, Dolan M C, Cramer C L. RTB Lectin: a novel receptor-independent delivery system for lysosomal enzyme replacement therapies. Sci Rep. 2015; 5:14144.
50. Acosta W, Devaiah S, Yang T, et al. RTB-lectin facilitates delivery of enzymes across the blood-brain-barrier in MPSI and MPSIIIA mice models. In: Dickson P, Clarke L, Bigger B W, cds. 15th International Symposium on MPS and Related Diseases. San Diego; 2018:18.
51. Condori J, Katta V, Acosta W, et al. Novel bioproduction and delivery strategies for MPS IIIA enzyme replacement therapeutics. Mol Genet Metab. 2016; 117 (2):S36.
52. Acosta W, Ayala J, Dolan M C, Cramer C L. RTB Lectin: A novel receptor-independent delivery system for lysosomal enzyme replacement therapies. Sci Rep. 2015; 5.
53. Acosta W, Martin R, Radin D N, Cramer C L. High-throughput imaging method for direct assessment of GM1 ganglioside levels in mammalian cells. Data Br. 2016; 6:1016-1022.
54. Stapleton M, Sawamoto K, Alméciga-Díaz C J, et al. Development of bone targeting drugs. Int J Mol Sci. 2017; 18(7):1345.
55. Tomatsu S, Montaño A M, Oikawa H, et al. Enzyme replacement therapy in newborn mucopolysaccharidosis IVA mice: Early treatment rescues bone lesions? Mol Genet Metab. 2015; 114(2): 195-202.
Altschul, S. F. et al. (1990) “Basic Local Alignment Search Tool” J. Mol. Biol. 215:402-410.
Altschul, S. F. et al. (1997) “Gapped BLAST and PSI-BLAST: A New Generation of Protein Database Search Programs” Nucl. Acids Res. 25:3389-3402.
Chen et al. (2010) BioTechniques 49:513-518.
Clancy, M. and Hannah, L. C. (2002) “Splicing of the maize Shl first intron is essential for enhancement of gene expression, and a T-rich motif increases expression without affecting splicing” Plant Physiol. 130(2):918-29.
Furtado, A. et al. (2002) “Tools for Use in the Genetic Engineering of Barley” Proceedings of the 10^{th} Australian Barley Technical Symposium, Canberra, ACT, Australia.
Good, X. et al. (1994) “Reduced ethylene synthesis by transgenic tomatoes expressing S-adenosylmethionine hydrolase” Plant Molec. Biol. 26:781-790.
Hwang, Y-S. et al. (2002) “Analysis of the Rice Endosperm-Specific Globulin Promoter in Transformed Rice Cells” Plant Cell Rep. 20:842-847.
Karlin S. and Altschul, S. F. (1990) “Methods for Assessing the Statistical Significance of Molecular Sequence Features by Using General Scoring Schemes” Proc. Natl. Acad. Sci. USA 87:2264-2268.
Karlin S. and Altschul, S. F. (1993) “Applications and Statistics for Multiple High-Scoring Segments in Molecular Sequences” Proc. Natl. Acad. Sci. USA 90:5873-5877.
Lewin, B. (1985) Genes II, John Wiley & Sons, Inc., p. 96.
Lovrinovic and Niemeyer (2005) BBRC 335:943-948.
Lungwitz et al. (2005) Eur. J. Pharmacet. Bioparmacet. 60:247-266.
Wu, C-L. et al. (1998) “Promoters of Rice Seed Storage Protein Genes Direct Endosperm-Specific Gene Expression in Transgenic Rice” Plant and Cell Physiology, 39(8):885-889.
Xu, D., McElroy, D., Thornburg, R. W., Wu, R. et al. (1993) “Systemic induction of a potato pin2 promoter by wounding, methyl jasmonate, and abscisic acid in transgenic rice plants” Plant Molecular Biology 22:573-588.
Bie et al. “Crystal structure analysis of human alpha-L-iduroni two crystal forms” GenBank accession number pbd/4JXP_A version GI: 480312357.
Scott et al. “Human alpha-L-iduronidase: cDNA isolation and expression” Proc. Natl. Acad. Sci. U.S.A. 88 (21), 9695-9699 (1991).
Van Damme et al. “Characterization and molecular cloning of Sambucus nigra agglutinin V (nigrin b), a GalNAc-specific type-2 ribosome-inactivating protein from the bark of elderberry (Sambucus nigra)” Eur. J. Biochem. 237 (2), 505-513 (1996).
Maveyraud et al. “Structural basis for sugar recognition, including the tn carcinoma antigen, by the lectin sna-ii from sambucus nigra” Proteins 75 p. 89 (2009).
Van de Kamp et al. “Genetic heterogeneity and clinical variability in the Sanfilippo syndrome (type A, B, and C)” Clin. Genet. 20 (2), 152-160 (1981).
Montfort et al. “The three-dimensional structure of ricin at 2.8A” J. Biol Chem. 262 (11), 5398-5403 (1987).
Citores L, Munoz R, Rojo M A, Jimenez P, Ferreras J M, Girbes T (2003) Cell. Molec. Biol. 49:461-465.
Citores L, Munoz R, De Benito F M, Iglesias R, Ferreras J M, Girbes T (1996) Cell. Molec. Biol. 42(4):473-476.
Sandvig K, van Deurs B (1999) FEBS Lett 452(1-2):67-70.
Simmons B M, Stahl P D, Russell J H (1986) J Biol Chem 261(17):7912-7920.
Van Damme et al., (1998) Crit. Rev. Plant Sci. 17: 575-692.
Bevan et al. “The structure and transcription start site of a major potato tuber protein gene” Nucleic Acid Res. 14 (11), 4625-4638 (1986).
Russell, C et al. “Murine MPS I: insights into the pathogenesis of Hurler syndrome.” Clinical genetics vol. 53,5 (1998): 349-61.

We claim:

1. A method for treating a musculoskeletal condition, the method comprising administering, to a subject in need thereof, (i) a fusion construct comprising a lectin and a therapeutic agent, (ii) a polynucleotide sequence encoding the fusion construct comprising the lectin and the therapeutic agent, (iii) a cell comprising the polynucleotide sequence encoding the fusion construct comprising the lectin and the therapeutic agent, or (iv) a cell expressing the fusion construction comprising the lectin and the therapeutic agent.

2. The method of claim 1, wherein the musculoskeletal condition is selected from genetic diseases with skeletal pathology, lysosomal diseases with skeletal pathology, collagen-associated diseases, bone diseases, and genetic muscle diseases.

3. The method of claim 1, wherein the musculoskeletal condition is selected from Muscular Dystrophy, Limb-Girdle, Autosomal Recessive 1; Glycogen Storage Disease; Muscular Dystrophy, Limb-Girdle, Autosomal Recessive 21; Ehlers-Danlos Syndrome; Muscular Dystrophy-Dystroglycanopathy; Autosomal Recessive Limb-Girdle Muscular Dystrophy Type 2a; Neuraminidase Deficiency; Niemann-Pick Disease, Type B; Mucopolysaccharidosis type I (MPS I); Mucopolysaccharidosis type II; Mucopolysaccharidosis type IIIA; Mucopolysaccharidosis type IIIB; Mucopolysaccharidosis type IIID; Mucopolysaccharidosis type IVA; Mucopolysaccharidosis type VI; Mucopolysaccharidosis type IX; Mannosidosis; Alpha B; Lysosomal; Fucosidosis; Schindler Disease, Type I; and Osteogenesis Imperfecta.

4. The method of claim 1, wherein the musculoskeletal condition is selected from MPS I, MPS IVA, MPS IVB, MPS VI, and POMPE Disease.

5. The method of claim 1, wherein the administration is oral, subcutaneous, intradermal, intravenous, intramuscular, intraperitoneal, intrajoint or intrasternal administration.

6. The method of claim 1, wherein the fusion construct comprises a plant lectin and a therapeutic protein.

7. The method of claim 1, wherein the lectin is a plant lectin.

8. The method of claim 7, wherein the plant lectin is selected from:
1) B subunits from AB toxins such as ricins, abrins, nigrins, and mistletoe toxins, viscumin toxins, ebulins, pharatoxin, hurin, phasin, and pulchellin; and
2) wheat germ agglutinin, peanut agglutinin, and tomato lectin.

9. The method of claim 8, wherein the plant lectin is RTB or NNB.

10. The method of claim 1, wherein the therapeutic agent is a therapeutic protein.

11. The method of claim 10, wherein the therapeutic protein is selected from enzymes, proteases, antibodies, chaperones, and growth factors.

12. The method of claim 10, wherein the therapeutic protein is selected from CAPN3, GYS1, POGLUT1, GAA, PYGM, AGL, GBE1, PYGL, PFKM, ALDOA, GYS2, ENO3, DSE, CHST14, PGAM2, GTG1, FKRP, POMT1, CAPN3, NEU1, SMPD1, IDS, NAGLU, SGSH, IDUA, GNS, GALNS, ARSB, HYAL1, MAN2B1, FUCA1, NAGA, CTSA, P3H1, FKBP10, BMP1, WNT1, SPARC, MESD, CRTAP, SERPINH1, SERPINF1 and TENT5A.

13. The method of claim 6, wherein the fusion construction is selected from IDUA:RTB, RTB:GALNS, GAA:RTB, CRTAP:RTB, P3H1:RTB, beta-Gal:RTB, and ARSB:RTB.

14. The method of claim 13, wherein the fusion construct is produced in plant, fungal, bacterial cells, mammalian cells or organisms.

15. The method of claim 6, wherein the plant lectin is fused to the therapeutic protein via a linker or a spacer sequence of amino acids.

16. The method of claim 1, wherein the polynucleotide sequence encoding the fusion construct comprising the lectin and the therapeutic agent is used as a gene therapy.

17. The method of claim 16, wherein the gene therapy is an adeno-associated virus (AAV) vector gene therapy.

18. A method for delivering a therapeutic agent to cells of the musculoskeletal system of a subject, the method comprising administering, to the subject, (i) a fusion construct comprising a lectin and a therapeutic agent, or (ii) a polynucleotide sequence encoding the fusion construct comprising the lectin and the therapeutic agent.

19. The method of claim 18, wherein the fusion construct comprises a plant lectin and a therapeutic protein.

20. The method of claim 18, wherein the fusion construct is selected from IDUA:RTB, RTB:GALNS, GAA:RTB, CRTAP:RTB, P3H1:RTB, beta-Gal:RTB, and ARSB:RTB.
